(19) United States
(12) Patent Application Publication
(10) Pub. No.: US 2025/0248721 A1
(43) Pub. Date: Aug. 7, 2025

(54) MEASURING DEVICES FOR LITHOTRIPSY CAPTURE SYSTEMS

(71) Applicant: GYRUS, ACMI,INC., D/B/A OLYMPUS SURGICAL TECHNOLOGIES AMERICA, Westborough, MA (US)

(72) Inventors: Kester Julian Batchelor, Mound, MN (US); David M. Leak, Brooklyn Park, MN (US); Charles A. Baker, Rogers, MN (US)

(21) Appl. No.: 19/042,845

(22) Filed: Jan. 31, 2025

Related U.S. Application Data
(60) Provisional application No. 63/627,932, filed on Feb. 1, 2024.

(51) Int. Cl. A61B 17/22

(52) U.S. Cl. CPC A61B 17/22; A61B 2017/22079

(57) ABSTRACT

A stone fragment capture device for a lithotripsy system comprises a container for retaining stone fragments, an inlet port in the container coupling to a suction passage of a lithotripsy device, an outlet port fluidly coupling to a collection canister of the lithotripsy system, a transparent portion of the container through which stone fragments are viewed and indicia on the container for determining a property of stone fragments. A method of retrieving stone fragments from a lithotripsy procedure comprises fragmenting stones with a lithotripsy device, drawing a vacuum through the lithotripsy device to pull stone fragments and waste fluid through the lithotripsy device, pulling the vacuum through a stone fragment capture device connected to the lithotripsy device, separating the stone fragments from the waste fluid within the stone fragment capture device, determining a property of the stone fragments within the stone fragment capture device, and finishing the lithotripsy procedure.

### PRIORITY CLAIM

This application claims the benefit of priority to U.S. Provisional Patent Application Ser. No. 63/627,932, filed Feb. 1, 2024, the contents of which are hereby incorporated by reference in their entirety.

### TECHNICAL FIELD

The present disclosure relates generally, but not by way of limitation, to medical devices that can be used to break obstructions, such as physiological calculi or “stones” using lithotripsy.

More specifically, but not by way of limitation, the present disclosure relates to systems, devices and methods for capturing stone fragments from lithotripsy systems.

### BACKGROUND

Medical endoscopes were first developed in the early 1800s and have been used to inspect inside the body. A typical endoscope includes a distal end comprising an optical or electronic imaging system and a proximal end with controls for manipulating tools and devices for viewing the image, with a solid or tubular elongate shaft connecting the ends. Some endoscopes allow a physician to pass tools or treatments down one or more hollow working channels, for example, to resect tissue or retrieve objects.

Over the past several decades, several advances have been made in the field of endoscopy, and in particular relating to the breaking up of physiologic calculi in the bile ducts, urinary tract, kidneys, and gall bladder. Physiological calculi in these regions may block ducts and cause a patient to experience a substantial amount of pain. Therefore, these calculi are typically broken down for surgical removal or biological passing. Different techniques and procedures have been developed to break up stones, including ultrasonic lithotripsy, pneumatic lithotripsy, electro-hydraulic lithotripsy (EHL), and laser lithotripsy including dissolution of calculi using green light, YAG, or holmium lasers.

### OVERVIEW

The present inventors have recognized, among other things, that problems to be solved in performing lithotripsy procedures is the recovery of stone fragments from the patient during or after the procedure. Typically, stone fragments are removed from inside the patient via suction applied to a distal end of a lithotripsy device. The suction pulls the stone fragments proximally through a suction tube and, typically, deposits the fragments into a waste container, along with other fluids from the patient, such as biological fluid and lavage fluid. The waste container is typically located away from the lithotripsy device, such as near a suction pump placed in the surgical area. As such, stone fragments typically comprise part of a mixture of materials located in a single container, thereby making retrieval of the stone fragments difficult. Retrieval of stone fragments can be desirable so that analysis can be performed. For example, doctors can view the stone fragments to prescribe diets to prevent future formation of stones or the stone fragments can be sent off to a laboratory for detailed composition analysis. Furthermore, it can be desirable to retrieve the stone fragments in order to evaluate whether or not all of the stones within the patient have been recovered.

The present subject matter can provide solutions to this problem and other problems by providing stone fragment capture devices and systems that collect stone fragments for separation from other collected material, such as waste fluids. The stone fragment capture devices of the present disclosure can retrieve stone fragments from the flow of waste fluid being removed from the patient via suction, while allowing the flow of waste fluid to pass through the stone fragment capture devices. The stone fragments can be retained in a container of the stone fragment capture device. The container can hold the stone fragments for later removal and analysis pre-separated from the waste fluid. As such, it is not necessary for personnel to manually strain the stone fragments from the waste container connected to the suction pump. In examples, the stone fragments can be stored in a sealed container for shipment to a laboratory without needing user intervention or repackaging.

The present inventors have also recognized, among other things, that problems to be solved in performing lithotripsy procedures is the difficulty in determining how much biological matter has been retrieved from a patient during the procedure. For example, it can be determined preoperatively that a patient has a particular type of stone formation using imaging, such as x-ray imaging. The number of stones in the formation and the size of each the stones can be estimated from the imaging. As such, a surgeon can estimate how much biological matter is to be collected from the patient. However, it can be difficult to determine intraoperatively if the stone fragments that have been recovered encompass all of the stones identified in the stone formation of the imaging. As mentioned, stone fragments are typically suctioned through the lithotripsy device into a waste fluid collection canister where the stone fragments are difficult to view. Collection and analysis of the stone fragments from the waste fluid collection canister would likely occur post-operatively due to the waste fluid collection canister being connected to the suction line being used during the procedure.

The present subject matter can provide solutions to this problem and other problems by providing stone fragment capture devices that can provide intraoperative analysis of collected stone fragments. The stone fragment capture devices of the present disclosure can facilitate intraoperative viewing of captured stone fragments, such as by including windows or by being fabricated of transparent material. Viewing of the captured stone fragments can allow for determination of the magnitude, e.g., volume or size, of captured stone fragments. The stone fragment capture devices of the present disclosure can include indicia to facilitate determining or estimating the volume or size of captured stone fragments, such as hash marks, graduation marks, scale marks, numeric values, text, graphics, symbols and the like. Furthermore, the stone fragment capture devices of the present disclosure can include multiple sets of indicia to facilitate obtaining information from the captured stone fragments in multiple orientations.

In an example, a stone fragment capture device for a lithotripsy system can comprise a container for retaining stone fragments, an inlet port in the container for coupling to a suction passage of a lithotripsy device, an outlet port for fluidly coupling to a collection canister of the lithotripsy system, a transparent portion of the container through which stone fragments can be viewed and indicia on the container for determining a property of stone fragments disposed within the container.

In another example, a lithotripsy device can comprise a handpiece configured to be held by a user, an energization source configured to generate an energy for breaking apart a physiological calculi, a shaft having a proximal end extending from the handpiece and a distal end configured to engage the physiological calculi, and a stone fragment capture device fluidly connected to the handpiece, the stone fragment capture device comprising a container into which waste fluid and stone fragments from the handpiece can flow, a trap element connected to the container to extract the stone fragments from flow of the waste fluid, and indicia provided on the stone fragment capture device for comparison to stone fragments within the stone fragment capture device.

In an additional example, a method of retrieving stone fragments from a lithotripsy procedure can comprise fragmenting stones with a lithotripsy device; drawing a vacuum through the lithotripsy device to pull stone fragments and waste fluid through the lithotripsy device, pulling the vacuum through a stone fragment capture device connected to the lithotripsy device, separating the stone fragments from the waste fluid within the stone fragment capture device, determining a property of the stone fragments within the stone fragment capture device, and finishing the lithotripsy procedure.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

FIG. 1 is a schematic illustration of an exemplary lithotripsy system with which the various stone fragment capture devices and systems of the present disclosure can be used.

FIG. 2 is a perspective view of a lithotripsy system suitable for use with the measuring devices of the present disclosure comprising a hand-held probe configured to deliver high frequency and ultrasonic energy for the fragmentation of stones.

FIG. 3 is a perspective view of a suction pump and a stone capture canister suitable for use with the lithotripsy system of FIG. 2.

FIG. 4 is a close-up perspective view of a handpiece of the hand-held probe of FIG. 2 in the hand of a user.

FIG. 5 is a schematic diagram of components of the lithotripsy system and suction system of FIG. 2 through FIG. 4 interacting with a kidney having stones.

FIG. 6 is a schematic cross-sectional illustration of a stone fragment capture device having volume indicators and that is configured for coupling directly to a handpiece of a lithotripsy device in an in-line orientation.

FIG. 7 is a schematic cross-sectional illustration of a stone fragment capture device having multiple volume indicators for reading in different orientations and that is configured for coupling directly to a handpiece of a lithotripsy device in an angled orientation.

FIG. 8A is a schematic cross-sectional illustration of a stone fragment capture device having volume indicators and that is configured for coupling directly to a handpiece of a lithotripsy device via a swivel coupling.

FIG. 8B is a perspective view of a first deformable valve for use with the stone fragment capture devices of the present disclosure.

FIG. 8C is a cross-sectional view of the first deformable valve of FIG. 8B showing a flow path configured to receive a tube stem and deflectable interlocking features to close the valve.

FIG. 8D is a perspective view of a second deformable valve suitable for use with the stone fragment capture devices of the present disclosure.

FIG. 8E is a cross-sectional view of the second deformable valve of FIG. 8E showing a tapered flow path configured to receive a tube stem and deflectable flap features to close the valve.

FIG. 9 is a schematic cross-sectional illustration of a stone fragment capture device having volume indicators and having a cap that is configured for coupling to a fluid suction line.

FIG. 10 is a schematic cross-sectional illustration of a stone fragment capture device having volume indicators and that is configured for coupling to a handpiece of a lithotripsy device via an orientation tubing.

FIG. 11 is a schematic cross-sectional illustration of a stone fragment capture device having volume indicators and that utilizes a concentric filter element.

FIG. 12 is a schematic cross-sectional illustration of a stone fragment capture device having volume indicators and that utilizes a gravity trap with a bypass.

FIG. 13 is a schematic cross-sectional illustration of a stone fragment capture device having volume indicators and that utilizes a helical trap.

FIG. 14 is a schematic cross-sectional illustration of a stone fragment capture device having volume indicators and that utilizes a vortex trap.

FIG. 15 is a block diagram illustrating a method for retrieving stone fragments from a lithotripsy procedure using the devices and stone fragment capture systems of the present disclosure that can incorporate feedback and measurement capabilities.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

The present disclosure provides examples of devices, systems and methods that can help address problems associated with capturing stone fragments during lithotripsy procedures. In particular, the present disclosure provides examples of devices, systems and methods that can be used to retrieve stone fragments from the collection process for real-time and intra-operative analysis, as well as be saved for later post-operative analysis. Typically, collection of stone fragments for later analysis can be challenging due to the stone fragments being simultaneously collected with other fluids of the procedure, thereby requiring subsequent processing, such as separating and repackaging. Benefits of the approaches described herein include, among other things, capturing stone fragments in a container that segregates the stone fragments from flowing waste fluid during a procedure, thereby reducing post-processing procedures and times. Segregating the stone fragments directly within the waste fluid flow can allow for real-time intra-operative analysis, such as to determine or estimate the volume and size of the captured stone fragments. The captured stone fragments can be analyzed with the measurement devices described herein to provide feedback as to how much fragmented stone material has been captured to determine, for example whether or not all of the stones identified pre-operatively have been collected. Thus, the chances of needing a follow-up procedure to collect unfragmented stones or stray stone fragments that remain within the patient can be reduced or eliminated. The stone fragment capture devices of the present disclosure can include measurement devices, such as indicia or a graduated scale, on a transparent window against which the captured stone fragments can be viewed. Furthermore, the stone fragment capture devices of the present disclosure can facilitate viewing of the captured stone fragments in multiple orientations to reduce the need for a user to having to intra-operatively reorient a lithotripsy device to obtain feedback, thereby reducing procedure times and operator fatigue.

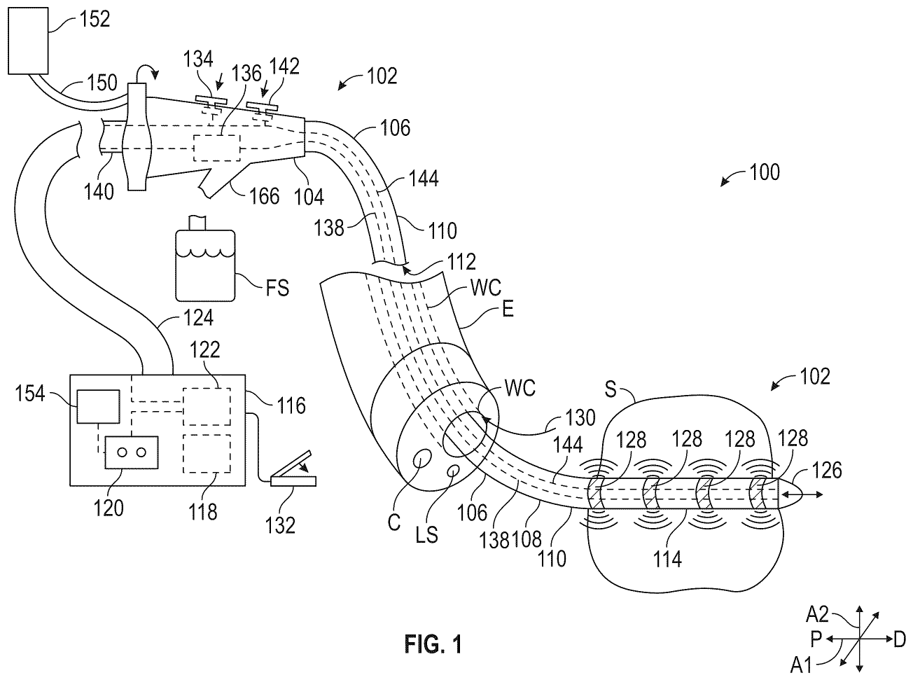

FIG. 1 illustrates an isometric view of an example of lithotripsy system 100 including lithotripter 102 having housing 104, such as a handle or handpiece. Lithotripter 102 can include delivery member 106 that is deliverable to a treatment site through working channel WC of endoscope E. Endoscope E can also include light source LS and camera C.

Delivery member 106 can include elongate shaft 108 having a tubular structure that can be flexible or rigid. Suitable materials for the delivery member include, but are not limited to, polytetrafluoroethylene (“PTFE”), polyethylenes (“PE”) and polyamides. Elongate shaft 108 can include outer surface 110 and at least one lumen 112 extending therethrough, the lumen being suitable for passage of components and materials that communicate with end effectors described herein.

Delivery member 106 can include an end effector such as probe 114 at a distal end that is deliverable to a treatment site. Probe 114 can be configured to deliver energy to fragment a mobile calculus such as a stone located in a bile duct, urinary tract, kidney or gall bladder. Probe 114 of lithotripter 102 can be introduced into a patient, driven by delivery member 106 through working channel WC of endoscope E or similar instrument. Probe 114 can be flexible or rigid.

Lithotripter 102 can be connected to signal generator 116. Signal generator 116 can include power source 118 or can be couplable to an external power source. Signal generator 116 can also include input 120 to receive an instruction from an operator, and can include controller 122 having processing circuitry for determining actions based on operator input and for sending control signals via output 124 for communication to lithotripter 102. Signal generator 116 can comprise an energization source that can produce signals and send the signals to probe 114 of lithotripter 102 to cause probe 114 to emit acoustic energy. Acoustic energy can include sound waves, sonic waves, ultrasonic waves or shock waves, or any combination of these. Acoustic energy can be delivered to stone S to deteriorate, crack and thereby fracture stone S. The examples herein are described with reference to combinations of ultrasonic and shock wave applications but any suitable acoustic energy, or combinations thereof, for fracturing stones can be provided. The terms sonic and ultrasonic may be used herein interchangeably, and can include any suitable acoustic energy for fragmenting stones. In additional examples, lithotripter 102 can be configured to deliver pneumatic, hydraulic or laser energy.

Features of probe 114 can provide improved fragmenting of stone S. For example, probe 114 can include drill 126 (which need not include a rotating drill bit), such as an ultrasonic drill that emits acoustic energy, in longitudinal direction A1, to drill a hole in the stone. In example, longitudinal direction A1 can extend in the proximal-distal, P-D, direction. Probe 114 can also include one or more lateral ultrasonic emitters 128 such as a lateral ultrasonic transducer to deliver acoustic energy inside the hole to fragment the stone from the inside out, such as by applying acoustic energy in radial or lateral direction A2. Lateral ultrasonic emitters 128 can emit ultrasonic energy radially relative to the axis of delivery member 106.

Drill 126 can be coupled to elongate shaft 108 and can be located at a distal tip of probe 114. Drill 126 can include at least a portion that extends distal of elongate shaft 108. In the example of FIG. 1, drill 126 can be configured to emit ultrasonic energy in longitudinal direction A1. Drill 126 can cause mechanical modification or destruction of stone S by producing pulsatile shock waves that move generally along longitudinal direction A1. Drill 126 can be configured to drill a hole, such as a recess into, or passage through, stone S. FIG. 1 shows an example including drill 126 that has drilled passage through stone S.

Drill 126 can be an ultrasonic emitter that receives ultrasonic energy from a remotely located ultrasonic drill transducer, which will be referred to as drill transducer 136 for the purposes of clarity over other emitters and transducers in this disclosure. Drill transducer 136 can be located, for example, in housing 104 of lithotripter 102. Drill transducer 136 can transmit ultrasonic energy in generally longitudinal direction A1, distally out of housing 104. Ultrasonic energy can be transmitted from drill transducer 136 to drill 126 via ultrasound transmission member 138. Ultrasound transmission member 138 can be coupled to drill transducer 136 at a proximal end and to drill 126 at the distal end. Ultrasound transmission member 138 can be formed of any material that is capable of transmitting the ultrasound energy from drill transducer 136 to drill 126, including but not limited to metal, metal alloys, shape memory alloys, polymers, ceramics, fibers, crystals or composites thereof.

Drill transducer 136 can be electrically couplable to signal generator 116, such as by connector 140, to receive signals for operation of drill 126. Drill transducer 136 can be actuated by, for example, an operator depressing foot pedal 132 that is in electrical communication with signal generator 116, or can be actuated by drill actuator 134 coupled to housing 104 that is in electrical communication with signal generator 116. Additionally, or alternatively, the drill transducer 136 may be operated based on the input 120 from the operator and/or the actions determined by the controller 122. Any other suitable actuator for controlling activation of drill 126 can be provided.

In addition to using an ultrasonic emitter for drilling, probe 114 can include at least one lateral ultrasonic emitter 128 configured to direct ultrasonic energy in radial or lateral direction A2, outward and away from longitudinal direction A1 such as toward an internal surface (e.g., an internal passage) of stone S. In the example of FIG. 1, the at least one lateral ultrasonic emitter 128 includes a plurality or array of lateral ultrasonic emitters 128.

Each of lateral ultrasonic emitters 128 can direct ultrasonic energy in lateral direction A2, with each of lateral ultrasonic emitters 128 located along a different longitudinal position on probe 114. In some examples lateral ultrasonic emitters 128 can be spaced apart along longitudinal direction A1. Lateral ultrasonic emitters 128 can extend laterally or radially around probe 114. In some examples, lateral ultrasonic emitters 128 can extend around the entire three-hundred-sixty-degree circumference of probe 114, or around a perimeter of probe 114 when a probe has a non-circular cross-section in direction lateral or perpendicular A1 to longitudinal direction A1. In other examples, lateral ultrasonic emitters 128 can only partially wrap around probe 114.

Lateral ultrasonic emitters 128 can be located proximal of drill 126. A benefit of this arrangement is that lateral ultrasonic emitter 128 can follow drill 126 so that after drill 126 prepares the internal passage in stone S, lateral ultrasonic emitter 128 can be advanced through the internal passage. When activated, such as by lateral emitter actuator 142 that is in electrical communication with lateral ultrasonic emitters 128 via electrical element 144 such as a wire, lateral ultrasonic emitters 128 can be configured to emit ultrasonic energy into to the internal passage and internal to stone S to fracture stone S from the inside of stone S.

Similar to drill transducer 136, lateral ultrasonic emitter 128 can include an ultrasonic or other acoustic transducer. An electrical-to-acoustic transducer is a component that can convert an electrical signal into variations in a physical quantity such as sound waves or pressure. Ultrasonic transducers can include linear piezoelectric stacks having piezoelectric elements located between two metal plates. In additional examples, magneto-restrictive stacks can be used. Such piezoelectric elements can convert electrical energy (e.g., electric current) into mechanical energy (e.g., sound waves, sonic waves, ultrasonic waves, shock waves). Piezoelectric elements can include crystal, such as quartz, having physical characteristics that results in the crystal undergoing mechanical stress when subjected to an electric field that causes the crystal to change size or shape. The piezoelectric elements or alternatively expand and contract in response to an alternating electric field, such as can be supplied by signal generator 116. This expansion and contraction can generate sound waves that can be delivered to stone S to fracture stone S.

To help locate stone S relatively stationary relative to working channel WC of endoscope E while drilling the hole, and relatively stationary to probe 114 (except for longitudinal A1 movement of probe 114 through the stone), suction 130, as denoted by an arrow, can be applied through working channel WC. Suction 130 can cause stone S to be “captured” by pulling stone S towards working channel WC and thus pulling stone S towards drill 126 of probe 114 for drilling. Upon fracturing of stone S, stone fragments can be suctioned into working channel WC.

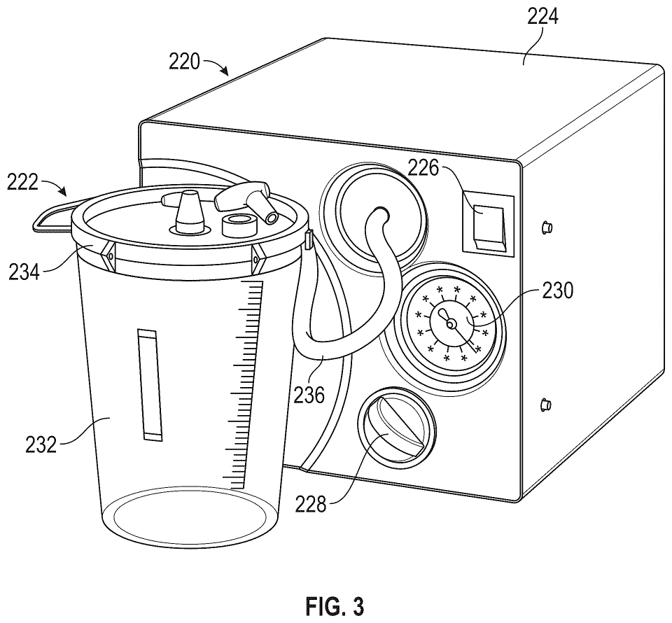

Some lithotripsy systems described herein can include fluid input 166 for receiving fluid from fluid storage FS and delivering fluid to a treatment site. For example, irrigation fluid or lavage fluid can be transmitted through endoscope E or elongate shaft 108. Typical stone fragment recovery systems involve simply collecting a mixture of solids and liquids retrieved from the patient while performing the procedure. For example, suction 130 can be applied at distal end of endoscope E or elongate shaft 108, and a vacuum drawn therethrough to deposit, materials, e.g., stone fragments and waste fluid, into a waste container. In examples, tube 150 can be connected to housing 104 to fluidly couple a lumen extending through working channel WC of endoscope E with collection container 152. Tube 150 can additionally be connected to suction device 154 or a pump to draw a vacuum through working channel WC, indicated by the arrow of suction 130, as explained in greater detail with reference to FIG. 3.

As discussed herein, the present disclosure provides stone capture fragment devices that can be positioned upstream of collection container 152 and downstream of lithotripter 102. In particular, the stone fragment capture devices of the present disclosure can be attached to lithotripter 102 or be positioned shortly after, e.g., downstream of, lithotripter 102. Thus, stone fragments can be collected before the stone fragments have an opportunity to obstruct or form a clog within tube 150. Furthermore, the stone fragment capture devices of the present disclosure can include capabilities for analyzing captured stone fragments. For example, the captured stone fragments can be measured to determine a volume or quantity of stone fragments collected within the stone fragment capture devices. Thus, with the present disclosure, lithotripsy system 100 can be equipped with a measurement system to provide feedback to a user as to the quantum of stone fragments collected within the stone fragment capture devices.

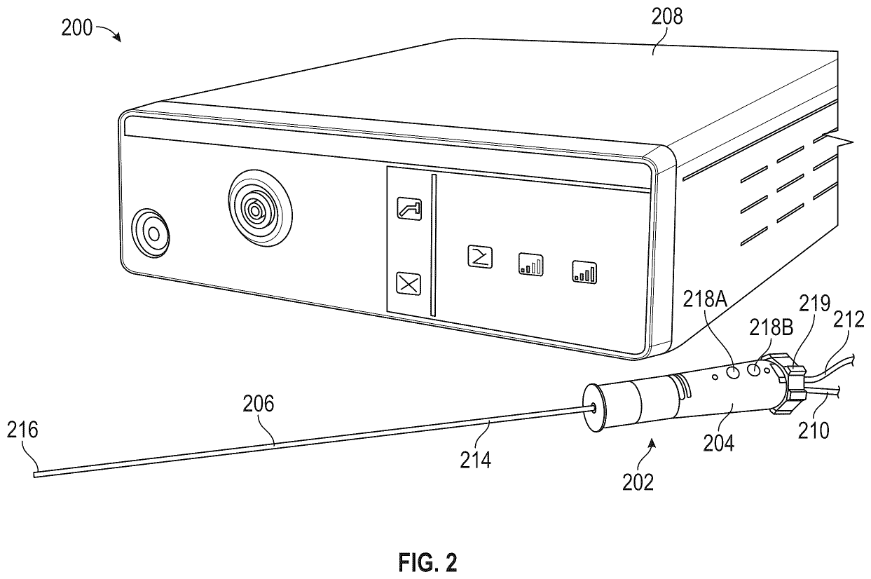

FIG. 2 is a perspective view of lithotripsy system 200 comprising hand-held probe 202 configured to deliver high frequency and ultrasonic energy for the fragmentation of stones. In examples, lithotripsy system 200 can comprise an oscillating lithotripter as is described in Pat. No. U.S. Pat. No. 9,974,552 to St. George et al. titled “Oscillating Lithotripter” and which is assigned to Gyrus ACMI, Inc., the contents of which is incorporated herein in its entirety. Features of the present disclosure can be added into lithotripsy system 200. Furthermore, lithotripsy system 200 can comprise an example of lithotripsy system 100 of FIG. 1.

Hand-held probe 202 can comprise handpiece or handle 204 and shaft 206. Hand-held probe 202 can be connected to generator console 208, such as via cable 210. Collection tube 212 can be connected to a storage container, such as fluid storage FS (FIG. 1) or container 232 (FIG. 3), to collect fluid and other biological material collected via shaft 206. Shaft 206 can extend from proximal end 214 to distal end 216 and can comprise an internal lumen. Handle 204 can further comprise button 218A and button 218B to control activation energy and knob 219 to control suction level.

FIG. 3 is a perspective view of suction pump 220 and stone fragment canister 222. Suction pump 220 can comprise housing 224, power switch 226, suction knob 228 and indicator 230. Stone fragment canister 222 can comprise container 232 and lid 234. Stone fragment canister 222 can be connected to suction pump 220 via tube 236.

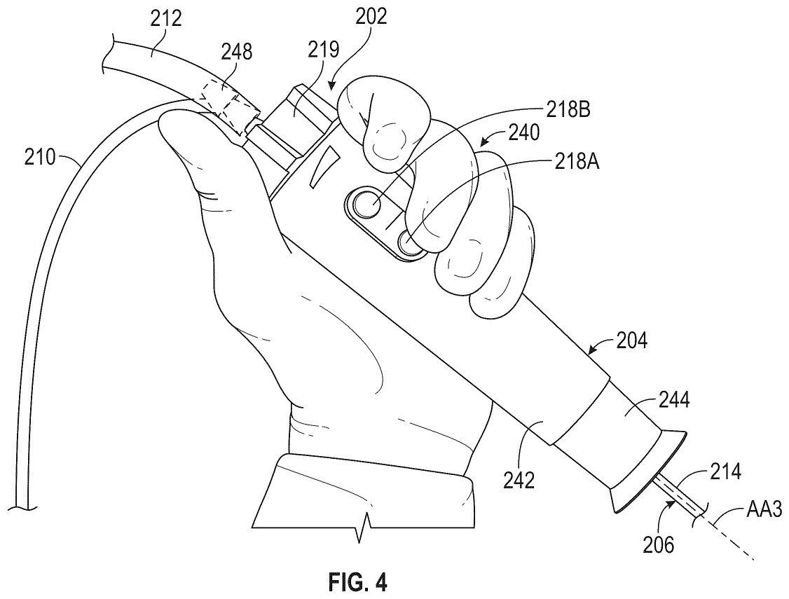

FIG. 4 is a perspective view of handle 204 in hand 240 of a user. Handle 204 can comprise handpiece 242. Knob 219 can be positioned at a proximal end of handpiece 242 and nosecone 244 can be positioned at a distal end of handpiece 242. Cable 210 and collection tube 212 can also be connected to a proximal end of handpiece 242.

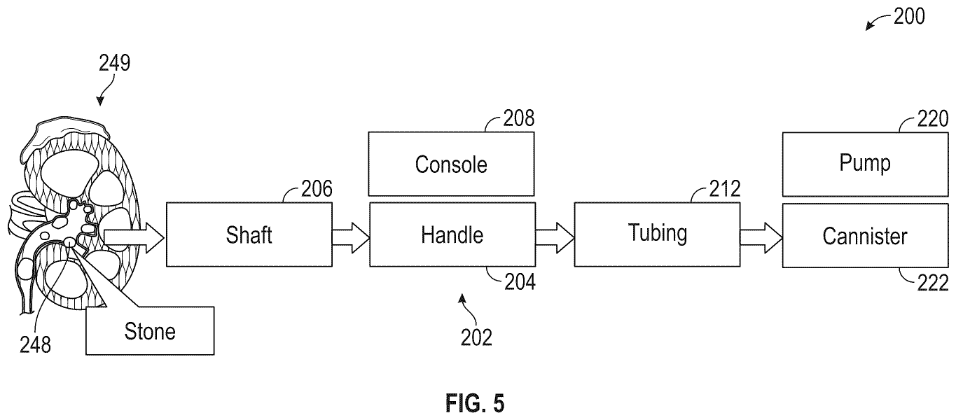

FIG. 5 is a schematic diagram of components of lithotripsy system 200 and suction pump 220 of FIG. 2 through FIG. 4 interacting with stone 248 of kidney 249. As discussed with reference to FIG. 3, FIG. 4 and FIG. 5, lithotripsy system 200 can comprise hand-held probe 202, suction pump 220 and stone fragment canister 222. Hand-held probe 202 can comprise shaft 206 and handle 204. Handle 204 can be connected to generator console 208 via cable 210. Handle 204 can be connected to stone fragment canister 222 via collection tube 212, and stone fragment canister 222 can be connected to suction pump 220.

FIG. 2 through FIG. 5 are discussed concurrently and provide disclosure of a lithotripsy system in which the stone fragment capture devices and measuring device of the present disclosure can be used. Lithotripsy system 100 of FIG. 1 and lithotripsy system 200 of FIG. 2 are examples of lithotripsy system that can be used with the stone fragment capture devices of the present disclosure, as well as the associated methods described herein. For example, a stone fragment measurement device of the present disclosure can include an indicator to inform a user of a quantity of stone fragments disposed within the stone fragment measurement device. In examples, the indicator can provide feedback related to the volume of stone fragments within the stone fragment capture device, such as through the use of a graduated scale provided on a wall or window of a container in which the stone fragments are captured. The stone fragment capture devices can be connected to housing 104 (FIG. 1) or handle 204 (FIG. 2) to provide a user with an indication of how much biological matter, e.g., stone fragments, is collected within the stone fragment capture devices.

With particular reference to FIG. 2, handle 204 can comprise any device suitable for facilitating manipulation and operation of shaft 206. Handle 204 can be located at proximal end 214 of shaft 206 or another suitable location along shaft 206. In examples, handle 204 can comprise a pistol grip, a knob, a handlebar grip and the like. In addition to or alternatively to buttons 218A and 218B and knob 219, handle 204 can comprise one or more of buttons, triggers, levers, knobs, dials and the like for control of energy activation, suction, irrigation and the like.

In various examples, distal end 216 of shaft 206, or another suitable location along shaft 206, can include a surgical device, which can comprise a component or device for interacting with a patient, such as those configured to cut and cauterize tissue and/or produce a desired tissue effect of the patient. In examples, the surgical tool can comprise forceps, a cutting tool, an ablation electrode, a cryogenic needle or applicator, an ultrasonic probe tip and the like, and combinations thereof. As such, hand-held probe 202 can be provided with a linkage, such as a mechanical linkage to actuate forceps or a cutting tool, an electrical linkage to activate an ablation electrode, an acoustic linkage, a liquid conduit (e.g., for the delivery of cryogenic argon gas) and the like, and combinations thereof. In examples, the surgical device can be included on a device used in conjunction with hand-held probe 202. In additional examples, hand-held probe 202 can comprise, or can be combined with, a device for viewing the patient, such as optical devices including endoscopes (e.g., endoscope E of FIG. 1) and fiberscopes.

Generator console 208 can comprise a source of energy for hand-held probe 202. For example, generator console 208 can be configured to provide electricity for performing ablation and cauterizing functions and/or ultrasonic energy for providing cutting, coagulating, fragmenting or other types of surgical functions. In examples, generator console 208 can provide ultrasonic wave energy, while intermittent ballistic shockwave energy is provided via an oscillating free mass within handle 204.

Shaft 206 can comprise an elongate member configured to deliver energy for fragmenting stones into a patient. Shaft 206 can be rigid and formed from a metal or plastic material. In examples, shaft 206 can be sized for performing lithotripsy procedures in conjunction with an endoscope. As such, shaft 206 can be inserted into an incision in the epidermis of a patient, through a body cavity of the patient and into an organ. Thus, it is desirable for the diameter or cross-sectional shape of shaft 206 to be as small as possible to facilitate minimally invasive surgical procedures. However, shaft 206 can also incorporate a lumen to allow for removal, e.g., via suction, of fragments of stones produced by the fragmentation energy. As such, the size of shaft 206 and a lumen extending therethrough must be balanced to allow for minimal invasiveness and adequate removal of stone fragments. For example, too small of a lumen can increase the time it takes to fragment the stones into suitably small pieces. However, removal of stone fragments can be provided by a lumen within a delivery scope, such as working channel WC of endoscope E of FIG. 1.

With particular reference to FIG. 3, lid 234 of stone fragment canister 222 can be connected to a suction line of a lithotripsy device, such as collection tube 212 of FIG. 2. Suction pump 220 can include a pump device within housing 224 to produce a vacuum within container 232 to draw liquid and stone fragments from a lithotripsy device into container 232. Liquid and stone fragments within collection tube 212 can enter into container 232. The liquid and stone fragments can be deposited on the bottom of container 232. A filter or trap device within container 232 can prevent stone fragments from passing through container 232. A plurality of instances of stone fragment canister 222 can be strung together in series to collect fluid and stone fragments volumes larger than container 232 can provide. A user can set the level, magnitude or amount of suction generated by suction pump 220 using suction knob 228. Indicator 230 can provide an indication of the amount of suction being generated. Power switch 226 can be used to turn on or turn off electrical power being provided to the pump device within suction pump 220.

Cable 210 can provide electrical power to, and electronic communication means with handle 204. For example, cable 210 can provide electrical power to transducers within shaft 206 or nosecone 244 to provide energy for breaking up stones. Cable 210 can also conduct ultrasonic energy. Button 218A and button 218B can control operation of the transducer within shaft 206 or nosecone 244, such as by providing differing activation levels, e.g., power, to the transducer.

Collection tube 212 can be connected to stem 246 on handpiece 242. Stem 246 and collection tube 212 can be in fluid communication with the interior of shaft 206. Suction from suction pump 220 can be pulled through collection tube 212, stem 246 and shaft 206. Knob 219 can be rotated to control the amount of suction provided to shaft 206.

With particular reference to FIG. 4, in operation, hand 240 of a user can grasp or hold handpiece 242 with the thumb positioned toward knob 219 and button 218A and button 218B positioned proximate the fingertips. As such, the thumb can be used to push knob 219 back and forth to adjust the suction level, while the fingertips can be used to adjust the transducer power level. Additionally, hand 240 can be moved distally, e.g., in the direction of shaft 206, to engage the tip of shaft 206 with a stone. As such, the user can simultaneously control the suction level and activation energy while pushing shaft 206 into the stone. A user can operate hand-held probe 202 by manipulating shaft 206 along axis AA3. Typically, a user can move handle 204 up and down with axis AA3 in a vertical orientation or back and forth with axis AA3 in a horizontal position. Otherwise, handle 204 can be held with axis AA3 in between vertical and horizontal. All throughout the procedure, or most of the procedure, hand 240 of the user is typically in close proximity to handle 204. With the present disclosure, stone fragment capture devices can be connected to handle 204, either directly or in close proximity thereto via a section of flexible tubing. As discussed herein, the stone fragment capture devices can separate stone fragments from waste fluid flowing out of handpiece 242. Due to the close proximity of the stone fragment capture devices to handpiece 242, the stone fragment capture device can be within easy view of the user of lithotripsy system 200 that is holding handpiece 242. As described herein, the stone fragment capture devices can include a container made of transparent material or that includes a window of transparent material that allows the captured stone fragments to be viewed within the container. The transparent material of the container can be located in one or more positions to allow the captures stone fragments to be viewed by the user for different orientations of handle 204. The stone fragment capture devices can be position in one or more orientations where at least one set of indicia is upright. Additionally, the transparent material can include thereon or in close proximity to indicia that can facilitate evaluation of the captured stone fragments. In examples, the indicia can include a graduated scale that provides an indication of the height of the collected stone fragments within the container. In examples, the indicia can include a graduated scale that provides an indication of the volume of the collected stone fragments within the container. In examples, the indicia can include a graduated scale that provides an indication of the size, e.g., diameter, of the collected stone fragments within the container. As such, the indicia can help a surgeon or other user evaluate the captured stone fragments to determine, for example, if a sufficient amount of stone fragments have been captured or if the stone fragments are being sufficiently fragmented.

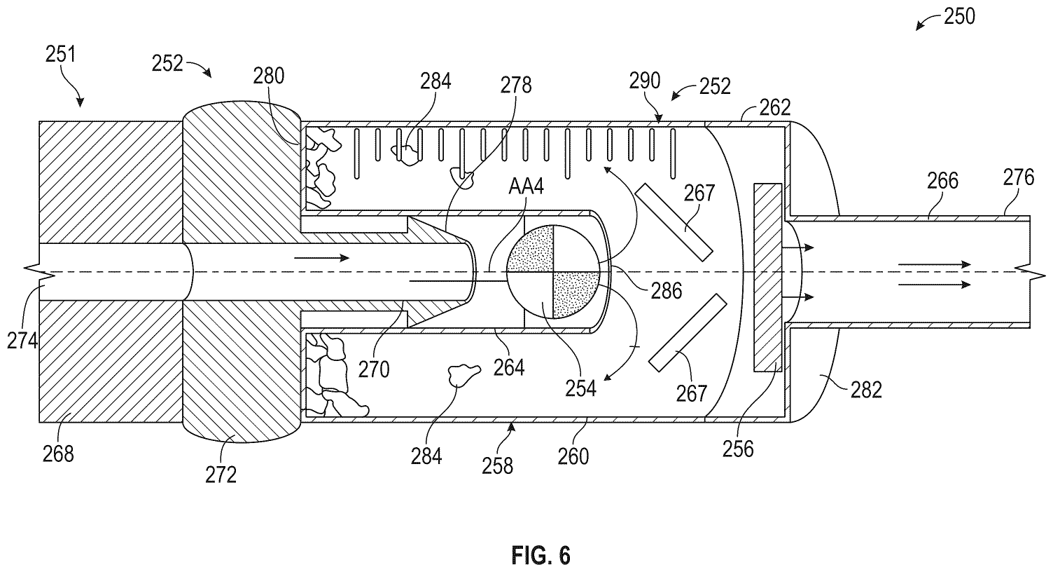

FIG. 6 is a schematic cross-sectional illustration of stone fragmentation system 250 comprising probe handpiece 251 and stone fragment capture device 252. Stone fragmentation system 250 can comprise any of the lithotripsy systems described herein, or another surgical system configured to generate suction therethrough. Stone fragmentation system 250 can be used with lithotripsy system 100 of FIG. 1 that can deliver fragmentation energy radially along probe 114, as well as lithotripsy system 200 of FIG. 2 that can deliver fragmentation energy longitudinally along shaft 206.

Stone fragment capture device 252 can comprise valve 254 and filter 256, which can be coupled to container 258. Container 258 can comprise housing 260, cap 262, inlet port 264 and outlet port 266. Probe handpiece 251 can comprise handle 204 of hand-held probe 202 (FIG. 2). Probe handpiece 251 can comprise handle 268 and stem 270. In examples, handle 268 can include knob 272 similar to knob 219 of FIG. 2 to control a function of stone fragmentation system 250, such as suction level. Passage 274 can be configured to extend from handle 268, such as from a shaft or probe extending distally therefrom, and through knob 272 and stem 270 located at a proximal end. In examples, knob 272 can be omitted or can be positioned at a distal end of handle 268.

Container 258 can be coupled to handle 268 and tube 276. Housing 260 can be positioned such that inlet port 264 couples to stem 270. Stem 270 can comprise barb 278 that can be configured to facilitate attachment of container 258 to handle 268. For example, barb 278 can be a resilient rim extending around stem 270 that can have a diameter slightly larger than the inner diameter of inlet port 264. Inlet port 264 can comprise a cylindrical tube extending from floor 280 of housing 260. A tube forming inlet port 264 can be tapered, e.g., inward toward axis AA4 moving from left to right in the orientation of FIG. 6, to slow movement of stone fragments 284 entering container 258. Floor 280 can comprise an annular disk that connects inlet port 264 to housing 260. Likewise, outlet port 266 can comprise a cylindrical tube extending from cap 262. Cap 262 can comprise end plate 282, which can comprise an annular disk that connects outlet port 266 to cap 262. Cap 262 can be coupled or fastened to housing 260 via any suitable means, such as a threaded connection or a snap fit connection. In examples, tube 276 can be integral with outlet port 266, as illustrated. In other examples, tube 276 can be coupled to outlet port 266 via a barbed connection similar to barb 278. Although the illustrated example shows container 258 being coupled directly to stem 270 of handle 268, container 258 can additionally be coupled to handle 268 via a length of tubing that can couple around stem 270 and be inserted into inlet port 264, for example.

Container 258 can be positioned between handle 268 and tube 276 to capture stone fragments 284 leaving passage 274. Stone fragments 284 can flow through passage 274 from handle 268 to stem 270. Stone fragments 284 can pass through inlet port 264 and be dispersed into the interior of container 258 within housing 260. Valve 254 can prevent stone fragments 284 from passing back into probe handpiece 251. Baffles 267 can deflect stone fragments 284 away from filter 256. Filter 256 can prevent stone fragments 284 from leaving container 258, but can permit fluid to flow out of container 258.

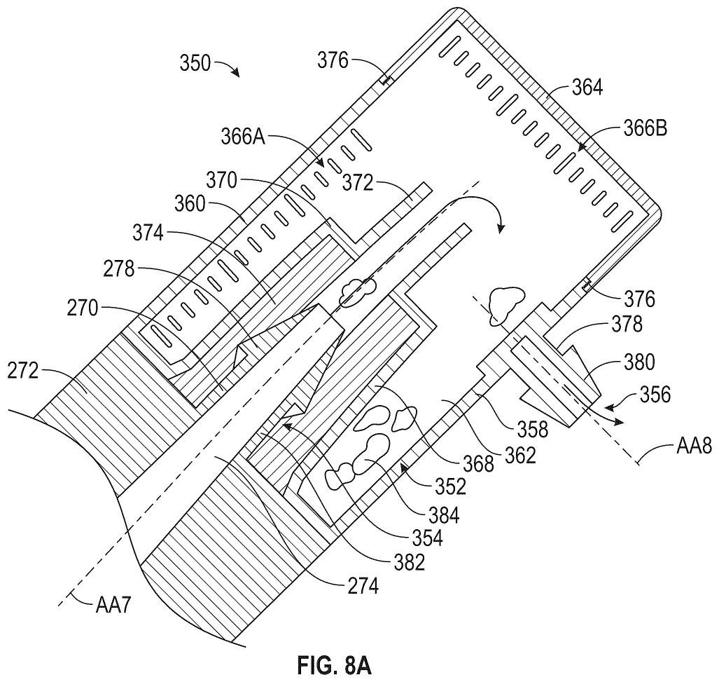

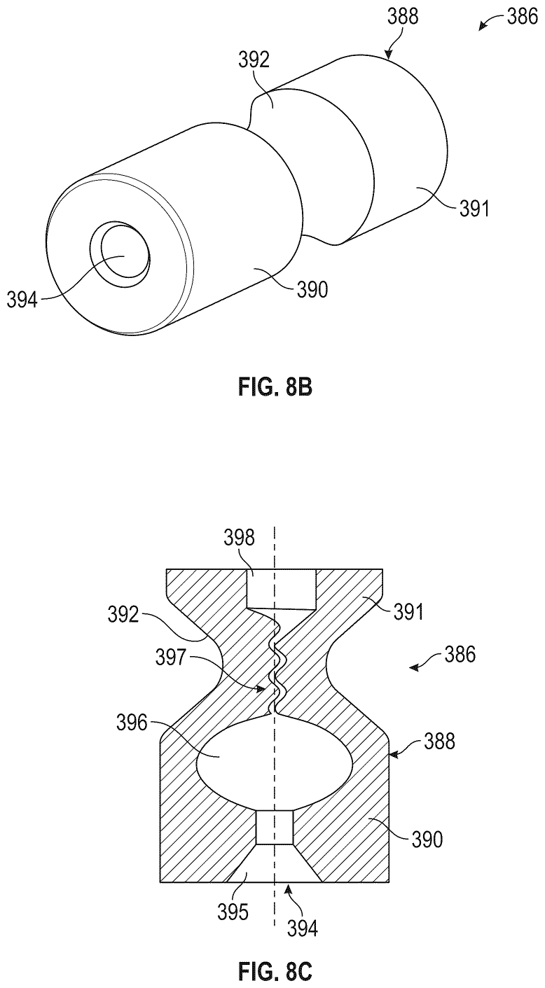

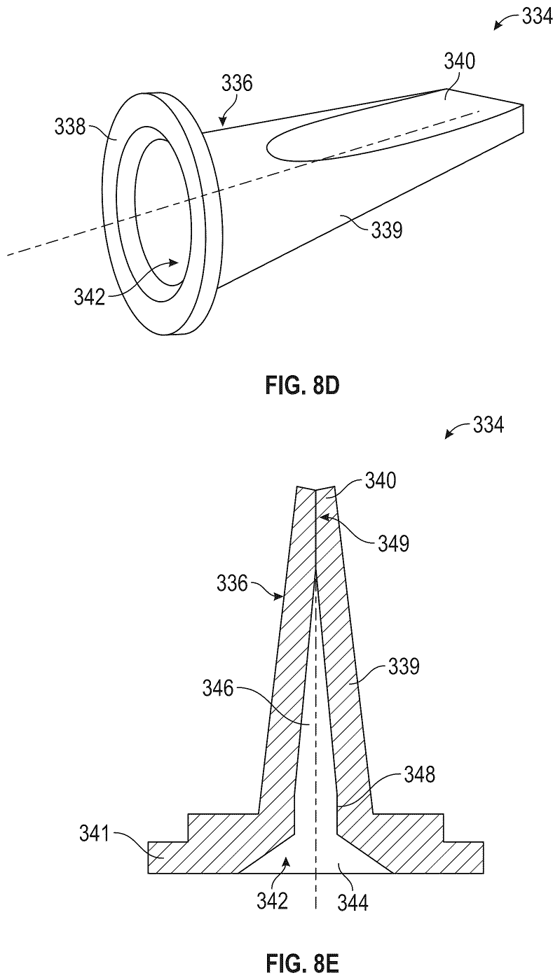

Inlet port 264 can be configured to align with outlet port 266 along axis AA4. However, in other examples, outlet port 266 can be offset from or angled relative to axis AA4, as discussed herein with reference to other examples or embodiments, e.g., FIG. 8A. One or more stone fragment capture elements, such as a tube comprising inlet port 264, valve 254, filter 256 and baffles 267 can be positioned on or in container 258 to facilitate retention of stone fragments 284 within container 258.

In examples, housing 260 can extend beyond tip 286 of inlet port 264 to provide clearance for stone fragments 284 to enter container 258. In examples, end plate 282 can be positioned a distance away from tip 286 to allow momentum of stone fragments 284 to dissipate. Additionally, baffles 267 can be positioned opposite of tip 286 to deflect stone fragments 284 into housing 260. Baffles 267 can comprise various shaped bodies extending radially inward from walls of housing 260 or axially outward from inlet port 264 to obstruct outlet port 266 from direct impingement of stone fragments 284. Baffles 267 can deaden the momentum of stone fragments 284 to facilitate movement of stone fragments 284 out of the suction path, e.g., path of flowing liquid, between inlet port 264 and outlet port 266.

Filter 256 can be positioned in container 258 to prevent egress of stone fragments 284 from container 258. In the illustrated example, filter 256 is positioned at outlet port 266. However, filter 256 can be positioned within outlet port 266 or anywhere else in container 258 to block free entry of material into outlet port 266. In examples, filter 256 can be mounted to cap 262 to facilitate access of stone fragments 284 within container 258 when cap 262 is removed. However, filter 256 can be configured to itself be removable from housing 260 independent of cap 262. Filter 256 can be sized to permit liquid and small pieces of tissue or stone fragments and other debris to pass through container 258, but that prevents large pieces of matter to be retained within container 258.

Valve 254 can be positioned proximate to tip 286 of stem 270 and can be used to intermittently close stem 270 to prevent egress of stone fragments 284 out of container 258. For example, when container 258 is removed from inlet port 264 and tube 276 is removed from cap 262, valve 254 can close to prevent stone fragments 284 from leaving container 258 at inlet port 264. Valve 254 can comprise any suitable device for allowing flow into housing 260 when container 258 is attached to stem 270 and preventing stone fragments from leaving housing 260 when container 258 is detached from handle 268. Valve 254 can be configured to be mechanically opened by engagement with stem 270 or by operation of a vacuum being pulled through container 258. Valve 254 can comprise a flapper valve, a ball valve, a biased stop or the like.

The capture elements described herein, such as valve 254, filter 256, baffles 267 and other filter or orifice elements, can be configured to retain pieces of matter or stone fragments 284 that are suitable for further analysis, such as visual viewing for quantity analysis, size analysis, color analysis and texture analysis. Container 258 can be provided with a feedback element or component, such as indicia 290, to allow the captured stone fragments to be analyzed and evaluated.

Stone fragments 284 can flow through passage 274 from handle 268 to stem 270. Stone fragments 284 can be dispersed into the interior of container 258. Baffles 267 can deflect stone fragments 284 radially away from axis AA4 to housing 260. Stone fragments 284 can fall downward due to the force of gravity. Stone fragments 284 can fall toward floor 280 if handle 268 is grasped by a user to position axis AA4 vertically. Stone fragments 284 can fall toward housing 260 if handle 268 is grasped by a user to position axis AA4 horizontally. Housing 260, floor 280 and end plate 282 can be fabricated of transparent material to allow stone fragments 284 to be viewed through container 258. Additionally, or alternatively, housing 260, floor 280 and end plate 282 can include a window fabricated of transparent material to allow stone fragments to be viewed within container 258. Multiple windows can be included to allow for viewing into container 258 from multiple orientations. In examples, the transparent material can comprise glass or plastic, such as polycarbonate.

Container 258 can be provided with markings to facilitate obtaining information from stone fragments 284. For example, indicia 290 can be disposed along housing 260 to provide one or more reference points against which stone fragments 284 within container 258 can be compared. In the illustrated example of FIG. 6, a single set of markings is provided on container 258. However, multiple sets of markings can be included on container 258 to, for example, allow for obtaining different types of information or for obtaining the same information in different orientations of container 258 during use. In examples, indicia 290 can provide an indication of a property of stone fragments 284, such as a length scale, a volume scale (e.g., zero at a first end and increasing volumes moving away from the first end), a size scale, a color scale (e.g., darker colors toward one end and lighter colors toward an opposite end with description of what each color means, such as a type of stone), a texture scale (e.g., a first pattern toward one end and a second pattern toward an opposite end with description of what each pattern means, such as a type of stone), a smoothness scale (e.g., a lighter density pattern toward one end and a darker density pattern toward an opposite end with description of what each density means, such as a type of stone,) and the like for comparing to stone fragments 284 within housing 260.

In examples, indicia 290 can comprise graduation marks that indicate a distance along housing 260. For example, the distance from floor 280 can be indicated with a scale similar to a ruler. Such graduation marks can facilitate determining a size, such as a diameter, of stone fragments 284. In examples, the axis of indicia 290 comprising a graduated scale can be aligned parallel with stem 270. As such, individual stone fragments 284 can be positioned adjacent to indicia 290, such as by orienting or shaking of stone fragment capture device 252, to facilitate analysis of individual stone fragments 284. In examples, indicia 290 can provide numeric or textual length indications such as inches or millimeters.

In examples, indicia 290 can comprise volume marks that indicate a volume of material above floor 280, similar to a graduated cylinder. The volume can be determined by taking the area of floor 280 and multiplying by the distance that each of the graduation marks is from floor 280. A user can position stone fragment capture device 252 such that floor 280 is positioned downward relative to gravity such that stone fragments 284 are resting on floor 280 and indicia 290 is upright. The user can shake stone fragment capture device 252 to settle stone fragments 284 closer to floor 280 and remove voids between adjacent to stone fragments 284. In examples, indicia 290 can provide numerical or textual volumetric indications, such as milliliters, cubic centimeters, cubic inches, fluid ounces or quarts.

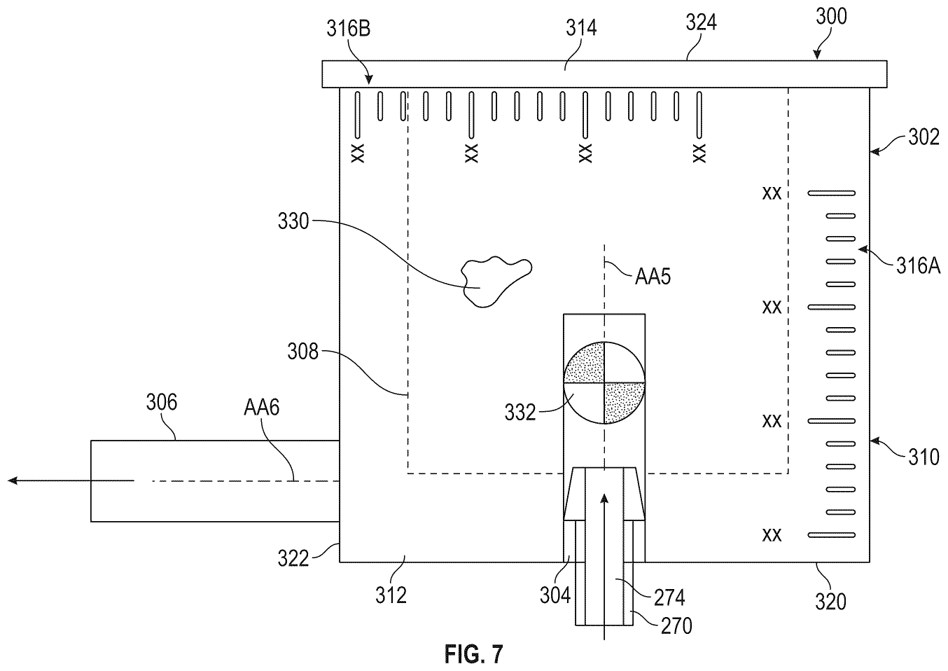

FIG. 7 is a schematic cross-sectional illustration of stone fragment capture device 300 suitable for use with any of the lithotripsy systems described herein, or another surgical system configured to generate suction therethrough. Stone fragment capture device 300 can be used with lithotripsy system 100 of FIG. 1 that can deliver fragmentation energy radially along probe 114, as well as lithotripsy system 200 of FIG. 2 that can deliver fragmentation energy longitudinally along shaft 206.

Stone fragment capture device 300 can comprise container 302, inlet socket 304, outlet stem 306 and filter 308. Container 302 can comprise housing 310 defining interior space 312 and cap 314. Container 302 can also include first indicia 316A and second indicia 316B.

Container 302 can be coupled to handle 268 (FIG. 6) at stem 270. Inlet socket 304 can be connected to stem 270 such that passage 274 can connect to the interior space 312 of housing 310. Inlet socket 304 can comprise a cylindrical opening extending through floor 320 of housing 310. Inlet socket 304 can comprise a resilient material having an opening slightly smaller than the diameter of stem 270 such that a tight, sealed fit can be achieved therebetween. Floor 320 can comprise an annular disk or polygonal plate that connects inlet socket 304 to housing 310. Likewise, outlet stem 306 can comprise a cylindrical tube extending from sidewall 322. Cap 314 can comprise end plate 324, which can comprise an annular disk connecting to sidewall 322. Cap 314 can be coupled or fastened to housing 310 via any suitable means, such as a threaded connection or a snap fit connection. In examples, collection tube 212 (FIG. 4) or tube 276 (FIG. 6) can be integral with or coupled to outlet stem 306 via a barbed connection similar to barb 278 (FIG. 6). Container 302 can be coupled directly to stem 246 of handpiece 242 (FIG. 4), but container 302 can also be coupled to handpiece 242 via a length of tubing that can couple around stem 246 and be inserted into inlet socket 304, for example.

Container 302 can be positioned between handle 268 (FIG. 6) and tube 276 (FIG. 6) to capture stone fragments 330 leaving passage 274. Stone fragments 330 can flow through passage 274 from handle 268 to stem 270. Filter 308 can be positioned in container 302 to prevent egress of stone fragments 330 from container 302. Filter 308 can comprise a mesh net or sock having mesh size smaller than the typical size of stone fragments 330 to prevent stone fragments 330 from flowing therethrough. Filter 308 can comprise a bag or sack attached to cap 314 and through which inlet socket 304 extends through a port or opening that can include a closure device such as a cinch strap or rubber band to hold filter 308 in close contact with inlet socket 304. As such, when cap 314 is removed from housing 310, filter 308 along with stone fragments 330 can be removed from container 302. In examples, filter 308 can be rotated relative to the orientation of FIG. 7 and attached to floor 320 such that inlet socket 304 extends into the open end of filter 308. Stone fragments 330 can be dispersed into interior space 312 of housing 310 within filter 308. In examples, housing 310 can extend beyond a tip of inlet socket 304 to provide clearance for stone fragments 330 to enter container 302. In examples, end plate 324 of cap 314 can be positioned a distance away from the tip of inlet socket 304 to allow momentum of stone fragments 330 to dissipate. Additionally, stone fragment capture device 300 can include baffles, such as baffles 267 of FIG. 6, to deflect stone fragments 330 into housing 310. Valve 332 can be positioned within inlet socket 304 and can be used to intermittently close inlet socket 304 to prevent egress of stone fragments 330 out of container 302. Valve 332 can comprise any suitable device for allowing flow into housing 310 when container 302 is attached to stem 270 and preventing stone fragments 330 from leaving housing 310 when container 302 is detached from stem 270. In examples, valve 332 can comprise a flapper valve, a ball valve, a biased stop or the like.

In the example of stone fragment capture device 300 of FIG. 7, inlet socket 304 can be configured to extend along axis AA5 and can be disposed at an angle relative to outlet stem 306 that extends along axis AA6. In the illustrated example, inlet socket 304 and outlet stem 306 are disposed at approximately a ninety-degree angle. However, in other examples, outlet stem 306 and inlet socket 304 can be disposed at other angles. Axis AA5 and axis AA6 can be disposed at different angles relative to the direction of gravity during operation of stone fragment capture device 300 depending on the orientation at which a surgeon is using the lithotripsy device attached to stone fragment capture device 300.

Stone fragments 330 can fall toward floor 320 if handle 268 (FIG. 6) is grasped by a user to position axis AA5 vertically relative to the orientation of FIG. 7. Stone fragments 330 can fall toward housing 310 if handle 268 (FIG. 6) is grasped by a user to position axis AA6 vertically. Housing 310, floor 320 and end plate 324 can be fabricated of transparent material to allow stone fragments to be viewed through container 302. Additionally, or alternatively, housing 310, floor 320 and end plate 324 can include a window fabricated of transparent material to allow stone fragments to be viewed within container 302. Multiple windows can be included to allow for viewing into container 302 from multiple orientations. In examples, the transparent material can comprise glass or plastic, such as polycarbonate. First indicia 316A and second indicia 316B can be positioned on or near transparent material to allow stone fragments 330 to be compared thereto. As with indicia 290 of FIG. 6, first indicia 316A and second indicia 316B can be used to determine, depth, volume, size, color and the like of stone fragments 330.

Container 302 can be provided with markings to facilitate obtaining information from stone fragments 330. For example, first indicia 316A can be disposed along housing 310 to provide one or more reference points against which stone fragments 330 within container 302 can be compared when axis AA5 is vertical, and second indicia 316B can be disposed along housing 310 to provide one or more reference points against which stone fragments 330 within container 302 can be compared when axis AA6 is vertical. In examples, the axis of first indicia 316A comprising a graduated scale can be aligned parallel with inlet socket 304, while the axis of second indicia 316B comprising a graduated scale can be aligned parallel with outlet stem 306.

In examples, first indicia 316A and second indicia 316B can comprise graduation marks that indicate a distance along housing 260. For example, the distance from floor 320 or housing 310 can be indicated with a scale similar to a ruler. Such graduation marks can facilitate determining a size, such as a diameter, of stone fragments 330. As such, individual stone fragments 330 can be positioned adjacent to first indicia 316A and second indicia 316B, such as by orienting or shaking of stone fragment capture device 300, to facilitate analysis of individual stone fragments 330.

In examples, first indicia 316A and second indicia 316B can comprise volume marks that indicate a volume of material above floor 320, similar to a graduated cylinder, or housing 310 in a similar fashion. For first indicia 316A, the volume can be determined by taking the area of floor 280 and multiplying by the distance that each of the graduation marks is from floor 280. For second indicia 316B, the volume can be determined by first calculating Area=cos⁻¹((r−h)/r) r²−(r−h) √(2rh−h²), where r is the radius of floor 320 and h is the level of fluid along housing 310 at one of the graduation marks of second indicia 316B, and then multiplying the length of housing 310 along which second indicia 316B extends. A user can position stone fragment capture device 300 such that floor 320 is positioned downward relative to gravity such that stone fragments 330 are resting on floor 320 to use first indicia 316A in an upright position. The user can shake stone fragment capture device 300 to settle stone fragments 330 closer to floor 320 and remove voids between adjacent to stone fragments 330. However, if it is inconvenient or uncomfortable for the user to orient floor 320 downward to use first indicia 316A in an upright position, the user can position housing 310 downward such that floor 320 and end plate 324 are vertical to use second indicia 316B. As such, stone fragment capture device 300 can allow a user to find the more comfortable position to orient stone fragment capture device 300 to analyze or assess stone fragments 330, thereby reducing the need for repositioning the lithotripsy device or having to remove the lithotripsy device from the patient to do so.

FIG. 8A is a schematic cross-sectional illustration of stone fragment capture device 350 suitable for use with any of the lithotripsy systems described herein, or another surgical system configured to generate suction therethrough. Stone fragment capture device 350 can be used with lithotripsy system 100 of FIG. 1 that can deliver fragmentation energy radially along probe 114, as well as lithotripsy system 200 of FIG. 2 that can deliver fragmentation energy longitudinally along shaft 206.

Stone fragment capture device 350 can comprise container 352, inlet stem 354, outlet stem 356 and filter 358. Container 352 can comprise housing 360 defining interior space 362 and lid 364. Container 352 can also include indicia 366A. Inlet stem 354 can comprise sidewall 368, end wall 370, neck 372 and insert 374. Lid 364 can be joined or fastened to sidewall 368 via threaded engagement 376. Outlet stem 356 can extend from sidewall 368 and can comprise neck 378 and barb 380. Stone fragments 384 can be positioned within container 352.

Stone fragment capture device 350 of FIG. 8A can be configured similarly as stone fragment capture device 252 of FIG. 6 except that outlet port 266 and tube 276 can be configured to extend from housing 260 rather than end plate 282, and inlet port 264 can include insert 374. Additionally, stone fragment capture device 350 can be configured to swivel about axis AA7. Insert 374 can comprise socket 382 that has a diameter that is slightly smaller than the diameter of stem 270. Insert 374 can be made of resilient material such that the material can be displaced by the presence of stem 270 within socket 382. Barb 278 can further displace the material of insert 374 to prevent axial displacement of stone fragment capture device 350 along axis AA7. However, the resiliency of insert 374 can be such that rotational movement of stone fragment capture device 350 about axis AA7 is not prevented. As such, stone fragment capture device 350 can rotate relative to handle 268 (FIG. 6) as a user manipulates the device. In other examples, other types of rotatable tube couplings can be used, such as a swivel connector for hoses and the like.

As discussed herein, container 352 can be fabricated of transparent material or can include one or more windows of transparent material. Indicia 366A can be located on or near the transparent material to allow stone fragments 384 to be compared to indicia 366A. As discussed herein, indicia 366A can comprise numerical information, textual information, symbols, or the like to allow for assessments and analysis of stone fragments 384 related to depth, volume, size, color and the like. In the illustrated example, a single set of indicia 366A is provided on container 352 for reading in a particular orientation or range of orientations of container 352. However, container 352 can include multiple sets of indicia to facilitate assessments and analysis of stone fragments 384 in multiple orientations. Container 352 can additionally include indicia 366B for providing the same information as indicia 366A in a different orientation. Alternatively, indicia 366B can be configured to provide different information from indicia 366A. For example, indicia 366A can be configured to provide volumetric information, while indicia 366B can be configured to provide color information.

FIG. 8B is a perspective view of deformable valve 386 for use with the stone fragment capture devices of the present disclosure. FIG. 8C is a cross-sectional view of deformable valve 386 of FIG. 8B showing a flow path configured to receive a tube coupler, such as a stem, and deflectable interlocking features to close the valve. FIG. 8B and FIG. 8C are discussed concurrently.

Deformable valve 386 can comprise main body 388 comprising inlet portion 390, outlet portion 391, indent 392 and fluid passage 394. Fluid passage 394 can comprise inlet 395, bulbous portion 396, interlocking section 397 and outlet 398.

Deformable valve 386 can be configured as an alternative to insert 374 of FIG. 8A. As such, deformable valve 386 can be configured and sized to be disposed within sidewall 368 of stone fragment capture device 350 of FIG. 8A. In additional examples, deformable valve 386 can be used in other stone fragment capture devices of the present disclosure, such as in place of valve 254 in FIG. 6, in place of valve 332 of FIG. 7 and in other places, such as those where a stem is or can be located.

Main body 388 can be fabricated from resilient material such as rubber, nitrile rubber, polymer and the like. Inlet portion 390 can be configured to receive a fluid input from a lithotripsy device. In examples, stem 270 (FIG. 8A) can be inserted into inlet portion 390 at fluid passage 394. Outlet portion 391 can be configured to abut end wall 370 (FIG. 8A) within stone fragment capture device 350 of FIG. 8A. Indent 392 can comprise an annular depression in main body 388 between inlet portion 390 and outlet portion 391. Indent 392 can allow for flexure of interlocking section 397, such as by comprising a thinned-down portion of main body 388 and by providing space for interlocking section 397 to deflect outward.

Inlet 395 can comprise a tapered entry portion to facilitate entry of stem 270 into fluid passage 394. Inlet 395 can additionally comprise a cylindrical portion fluidly connecting to bulbous portion 396. Bulbous portion 396 can provide space within main body 388 for receiving stem 270. Bulbous portion 396 can additionally help form thinned-down portions of main body 388 proximate to indent 392 to facilitate flexing of main body 388 at interlocking section 397. Inlet 395 can be narrower than bulbous portion 396 to provide a tight seal around stem 270. Interlocking section 397 can comprise opposing faces having interlocking features such as ridges, channels and the like that can form a circuitous path therebetween. Thus, when the opposing faces of interlocking section 397 abut one another, they can form a seal to prevent fluids from passing therebetween can be formed. The opposing faces of interlocking section 397 can be configured to abut one another at rest or in a non-deformed or non-stretched state. Outlet 398 can comprise a cylindrical passage fluidly coupled to interlocking section 397. Thus, inlet 395 can be fluidly closed-off from outlet 398 when interlocking section 397 is closed and can be fluidly connected to outlet 398 when interlocking section 397 is open.

In the state illustrated in FIG. 8C, such as when stem 270 is not inserted into inlet 395, the opposing faces of interlocking section 397 can push against each other to prevent fluid flow through deformable valve 386. Stem 270 can be inserted into inlet 395 and can be pushed into interlocking section 397 to push opposing faces of interlocking section 397 away from each other. As such, fluid leaving stem 270 can flow into outlet 398. Thus, fluid can pass through deformable valve 386. When stem 270 is removed from deformable valve 386 after a procedure, stone fragments 384 within interior space 362 (FIG. 8A) are inhibited from exiting stone fragment capture device 350 through deformable valve 386. In examples, another instance of deformable valve 386 can be positioned on outlet stem 356 (FIG. 8A) to prevent fluid from exiting therethrough.

FIG. 8D is a perspective view of deformable valve 334 suitable for use with the stone fragment capture devices of the present disclosure. FIG. 8E is a cross-sectional view of deformable valve 334 of FIG. 8D showing a tapered flow path configured to receive a tube coupler, such as a stem, and deflectable flap features to close the valve. FIG. 8D and FIG. 8E are discussed concurrently.

Deformable valve 334 can comprise main body 336 comprising inlet rim 338, tapered tube 339, outlet end 340 and fluid passage 342. Fluid passage 342 can comprise inlet 344 and tapered portion 346.

Deformable valve 334 can be configured as an alternative to insert 374 of FIG. 8A. As such, deformable valve 334 can be configured and sized to be disposed within sidewall 368 of stone fragment capture device 350 of FIG. 8A. In additional examples, deformable valve 334 can be used in other stone fragment capture devices of the present disclosure, such as in place of valve 254 in FIG. 6, in place of valve 332 of FIG. 7 and in other places, such as those where a stem is or can be located.

Main body 336 can be fabricated from resilient material such as rubber, nitrile rubber, polymer and the like. Inlet rim 338 can be configured to receive a fluid input from a lithotripsy device. In examples, stem 270 (FIG. 8A) can be inserted into inlet rim 338 at fluid passage 342. Inlet rim 338 can be configured to abut the bottom of housing 360 opposite lid 364 of stone fragment capture device 350 of FIG. 8A. Outlet end 340 can be configured to extend into or in close proximity to neck 372 (FIG. 8A). Tapered tube 339 can comprise a cylindrical tube proximate to inlet rim 338 that can flatten out at outlet end 340. Opposing walls of tapered tube 339 can abut each other at outlet end 340 to form a valve therebetween. In examples, deformable valve 334 can be configured as or similarly to a duckbill valve.

Inlet 344 can comprise a tapered entry portion to facilitate entry of stem 270 into fluid passage 342. Inlet 344 can additionally comprise cylindrical portion 348 fluidly connecting to tapered portion 346. Inlet 344 and tapered portion 346 can be sized to provide space within main body 336 for receiving stem 270. Main body 336 can comprise a thin-walled body to facilitate flexing at outlet end 340. Cylindrical portion 348 of inlet 344 can form a neck to provide a tight seal around stem 270. Outlet end 340 can comprise opposing faces 349 that abut one another, thereby forming a seal to prevent fluids from passing therebetween. Opposing faces 349 of outlet end 340 can be configured to abut one another at rest or in a non-deformed or non-stretched state. When opposing face 349 are opened, they can form a cylindrical passage fluidly coupled to inlet 344. Thus, fluid within inlet 34 can be prevented from leaving deformable valve 334 when opposing faces 349 are closed and can be allowed to leave deformable valve 334 when opposing faces are open.

In the state illustrated in FIG. 8E, such as when stem 270 is not inserted into inlet 344, opposing faces 349 of tapered portion 346 can push against each other at outlet end 340 to prevent fluid flow through deformable valve 334. Stem 270 can be inserted into inlet 344 and can be pushed into tapered portion 346 to push opposing faces 349 away from each other. As such, fluid leaving stem 270 can flow all the way through tapered portion 346 and exit outlet end 340. Thus, fluid can pass through deformable valve 3334. When stem 270 is removed from deformable valve 334 after a procedure, stone fragments 384 within interior space 362 (FIG. 8A) are inhibited from exiting stone fragment capture device 350. In examples, another instance of deformable valve 334 can be positioned on outlet stem 356 (FIG. 8A) to prevent stone fluid from exiting therethrough.

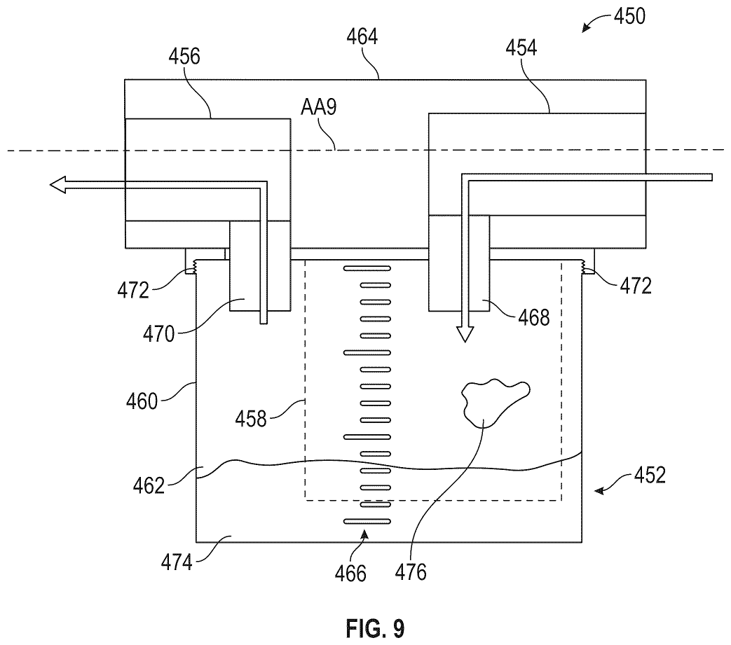

FIG. 9 is a schematic cross-sectional illustration of stone fragment capture device 450 suitable for use with any of the lithotripsy systems described herein, or another surgical system configured to generate suction therethrough. Stone fragment capture device 450 can be used with lithotripsy system 100 of FIG. 1 that can deliver fragmentation energy radially along probe 114, as well as lithotripsy system 200 of FIG. 2 that can deliver fragmentation energy longitudinally along shaft 206.

Stone fragment capture device 450 can comprise container 452, inlet socket 454, outlet socket 456 and filter 458. Container 452 can comprise housing 460 defining interior space 462 and lid 464. Container 452 can also include indicia 466. Inlet socket 454 can comprise first passage 468 and outlet socket 456 can comprise second passage 470. Lid 464 can be joined to housing 460 via threaded engagement 472. Waste fluid 474 and stone fragments 476 can be positioned within container 452.

Stone fragment capture device 450 can be configured to allow for stem 246 of handpiece 242 (FIG. 4) and for collection tube 212 (FIG. 4) to both be attached to lid 464. Thus, stem 246 can be inserted into inlet socket 454 and collection tube 212 can be inserted into outlet socket 456. First passage 468 can turn the flow of material entering inlet socket 454 toward container 452 and second passage 470 can fluidly connect container 452 with outlet socket 456. Thus, stone fragments 476 can flow into stone fragment capture device 450 at inlet socket 454, flow through first passage 468 and enter filter 458 within container 452. Filter 458 can comprise a bag or sock of mesh material to allow flow to pass through filter 458 and continue through second passage 470 and outlet socket 456. Thus, inlet socket 454 and outlet socket 456 can extend along axis AA9 that is axially aligned along axis AA3 of handpiece 242 (FIG. 4), but indicia 466 can be perpendicular to axis AA9 and handpiece 242. Thus, stone fragment capture device 450 can be conducive for procedures where hand-held probe 202 (FIG. 4) is used in a side-to-side manner with axis AA3 and axis AA9 being horizontal or generally horizontal.

Stone fragment capture device 252 of FIG. 6, stone fragment capture device 300 of FIG. 7, stone fragment capture device 350 of FIG. 8A and stone fragment capture device 450 of FIG. 9 can be configured similarly to each other to allow a user to determine a depth, volume, size, color and the like of stone fragments within a container. In particular, stone fragment capture device 252, stone fragment capture device 300, stone fragment capture device 350, and stone fragment capture device 450 can comprise housings (such as housing 260 of FIG. 6, housing 310 of FIG. 7, housing 360 of FIG. 8A and housing 460 of FIG. 9), e.g., rigid or flexible containers, that attach directly to handle 204 without an intervening length of tube. As such, stone fragments leaving handle 204 can directly enter stone fragment capture device 252, stone fragment capture device 300, stone fragment capture device 350 and stone fragment capture device 450. The housings can include a filter element to trap stone fragments within the housings while liquid material can pass through the housings. The housings can be opened to allow for access to and removal of the stone fragments.

The housings illustrated have been described as being circular cylindrical bodies, but can additionally comprise rectangular bodies, hexagonal bodies, e.g., a hexagonal prism, an octagonal body, e.g., an octagonal prism, or other shapes.

Stone fragment capture device 252 and stone fragment capture device 450 can allow for tubing, such as collection tube 212 (FIG. 4), to extend in the same direction as stem 246, e.g., axially from handle 204. Stone fragment capture device 252 can have indicia aligned in the same direction. Stone fragment capture device 450 can have indicia angled thereto. Stone fragment capture device 300 and stone fragment capture device 350 can allow for tubing, such as collection tube 212 (FIG. 4), to extend in different directions as stem 246, e.g., at an angle to or radially from handle 204. Stone fragment capture device 300 and stone fragment capture device 350 can have indicia aligned and angled thereto.

The housings can be fixedly attached to handle 204 such that the housings are immobilized. The housing can also be moveably attached to handpiece 242 such that the housing can rotate to reposition collection tube 212 to change circumferential position. The housings can be user attachable and detachable from handle 204. The housings can be integrated with handle 204, e.g., monolithic with, so as to not be user detachable.

Indicia can be provided on the housing of the stone fragment capture devices in one or more orientations to allow the volume to be determined in more than one orientation of the stone fragment capture device. The indicia can comprise visual markings, e.g., numbers, text, symbols and the like, to facilitate determination of a volume of captured stone fragments or size of captured stone fragments.

The features of stone fragment capture device 252, stone fragment capture device 300, stone fragment capture device 350 and stone fragment capture device 450 can be interchanged to achieve other combinations of features than those illustrated in FIG. 6, FIG. 7, FIG. 8A and FIG. 9.

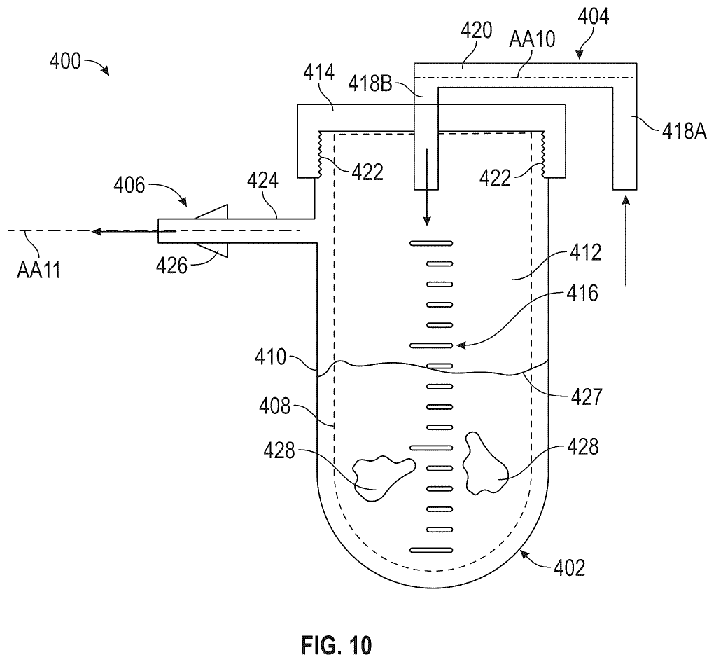

FIG. 10 is a schematic cross-sectional illustration of stone fragment capture device 400 suitable for use with any of the lithotripsy systems described herein, or another surgical system configured to generate suction therethrough. Stone fragment capture device 400 can be used with lithotripsy system 100 of FIG. 1 that can deliver fragmentation energy radially along probe 114, as well as lithotripsy system 200 of FIG. 2 that can deliver fragmentation energy longitudinally along shaft 206.

Stone fragment capture device 400 can comprise container 402, inlet stem 404, outlet stem 406 and filter 408. Container 402 can comprise housing 410 defining interior space 412 and lid 414. Container 402 can also include indicia 416. Inlet stem 404 can comprise first leg 418A, middle portion 420 and second leg 418B. Lid 414 can be joined to housing 410 via threaded engagement 422. Outlet stem 406 can extend from housing 410 and can comprise neck 424 and barb 426. Waste fluid 427 and stone fragments 428 can be positioned within container 402.

First leg 418A can be connected to handpiece 242 at stem 246 (FIG. 4) such as by using a barbed hose coupler, as described herein. Second leg 418B can attached to lid 414 either in a fixed manner or a rotational manner. Middle portion 420 can extend between first leg 418A and second leg 418B along axis AA10. As discussed below, at least one of first leg 418A, second leg 418B and middle portion 420 can be flexible to allow the orientation of stone fragment capture device 400 to be variable relative to handpiece 242. However, inlet stem 404 can be pre-curved or pre-shaped such that first leg 418A and second leg 418B are perpendicular to or angled relative to axis AA10 in a U-shape. First leg 418A, second leg 418B and middle portion 420 are shown being connected at right angles, but can also be connected by curved or rounded segments to form a smooth path therethrough. Inlet stem 404 can be configured to enter stone fragment capture device 400 through lid 414 and exit stone fragment capture device 400 through housing 410 at outlet stem 406 along axis AA11. In the illustrated example, outlet stem 406 is positioned near the top of housing 410. It can be advantageous for outlet stem to be positioned near the top of housing 410 to prevent stone fragments 428 from blocking outlet stem 406. However, outlet stem 406 can be positioned to exit the bottom portion of housing 410 or the bottom of housing 410 opposite to inlet stem 404 to facilitate draining of waste fluid 427 from housing 410. It can be advantageous to have stone fragments 428 enter stone fragment capture device 400 through lid 414 so that stone fragments 428 accumulate at the bottom of housing 410, thereby aligning with indicia 416. In the illustrated example, housing 410 is shown having a curved bottom, but can have a flat bottom to facilitate accumulation of stone fragments 428 in an even or consistent manner relative to indicia 416.

Stone fragment capture device 400 can be configured similarly to stone fragment capture devices of FIG. 6, FIG. 7, FIG. 8A and FIG. 9 in that a housing having a filter element and indicia can be attached to a lithotripter handle to capture stone fragments in a container that allows for viewing of the captured stone fragments relative to the indicia to determine the depth, volume, size, color or other parameters of captured stone fragments. However, stone fragment capture device 400 can be coupled to handle 204 in a variable manner to allow container 402 to be positioned into a plurality of different orientations. In particular, all or a portion of inlet stem 404 can be configured to bend and/or flex to allow stone fragment capture device 400 to be positioned in different orientations relative to handpiece 242 (FIG. 4).

In examples, one or more of first leg 418A, second leg 418B and middle portion 420 can comprise flexible tubing. Inlet stem 404 can bend and/or flex as a user manipulates handpiece 242 (FIG. 4) during a procedure. Thus, the bottom of housing 410 can remain oriented downward relative to gravity during use thereby facilitating stone fragments 428 aligning with indicia 416 and indicia 416 being in an orientation for reading by the user. For example, if handpiece 242 were being used so that axis AA3 (FIG. 4) connected to first leg 418A were vertical, stone fragment capture device 400 can also remain vertical (as shown in the orientation of FIG. 10) so that indicia 416 is vertical due to, for example, the pre-curved shape of first leg 418A and second leg 418B relative to middle portion 420. Thus, the pre-curvature of inlet stem 404 can be sufficiently strong to hold inlet stem 404 in the pre-curved U-shape. However, if handpiece 242 were being used so that axis AA3 (FIG. 4) were horizontal, inlet stem 404 can flex so that stone fragment capture device 400 can remain vertical (as shown in the orientation of FIG. 10) so that indicia 416 is vertical. Thus, inlet stem 404 can be sufficiently flexible to allow inlet stem 404 to bend out of the pre-curved U shape.

One of the benefits of the stone fragment capture devices of the present disclosure is the ability of stone fragments to flow directly into the stone fragment capture devices, as described, to avoid the potential of tubing, e.g., collection tube 212 (FIG. 4), becoming clogged. To reduce or eliminate the chances of inlet stem 404 becoming clogged, inlet stem 404 can be made of material that is sufficiently supple to allow for flexing, but that is sufficiently rigid to prevent collapsing, such as due to bending or the suction being drawn therethrough. In examples, first leg 418A, second leg 418B and middle portion 420 can be made of plasticized Polyvinyl Chloride (PVC). In examples, first leg 418A and middle portion 420 can comprise rigid tubes, and second leg 418B can comprise a flexible tube. In examples, first leg 418A and second leg 418B can comprise rigid tubes and middle portion 420 can comprise a flexible tube. Furthermore, inlet stem 404 is short thereby reducing the changes of stone fragments becoming lodged therein. In examples, the total length of inlet stem 404, including first leg 418A, second leg 418B and middle portion 420 can be less than approximately ten inches (˜25.4 cm). In examples, the diameter of inlet stem 404 can be larger than collection tube 212 (FIG. 4) to prevent blockage from stone fragments 428. In examples, the wall thickness of one or more of first leg 418A, second leg 418B and middle portion 420 can be thicker than the others and/or thicker than collection tube 212 (FIG. 4).

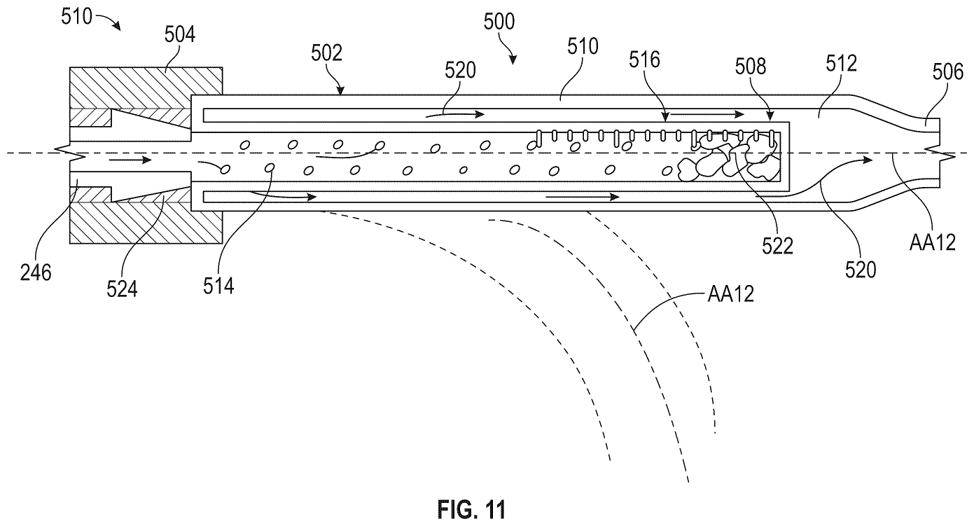

FIG. 11 is a schematic cross-sectional illustration of stone fragment capture device 500 suitable for use with any of the lithotripsy systems described herein, or another surgical system configured to generate suction therethrough. Stone fragment capture device 500 can be used with lithotripsy system 100 of FIG. 1 that can deliver fragmentation energy radially along probe 114, as well as lithotripsy system 200 of FIG. 2 that can deliver fragmentation energy longitudinally along shaft 206.

Stone fragment capture device 500 can comprise tube 502, coupler 504, outlet stem 506 and filter 508. Tube 502 can comprise elongate body 510 defining interior space 512 and openings 514. Tube 502 can also include indicia 516. Stone fragment capture device 500 can further comprise coupler 504 that can be used to attach to stem 270 of handle 268 (FIG. 6), for example. Waste fluid 520, as indicated by arrows, and stone fragments 522 can be positioned within tube 502.

Coupler 504 can comprise a cylindrical body having socket 524. Stem 246 of handpiece 242 (FIG. 4) can be inserted into socket 524 to couple stone fragment capture device 500 to handle 268. Coupler 504 can comprise a body that is integral with elongate body 510 or can be attached thereto by an interference fit, adhesive or the like. Outlet stem 506 can be attached to elongate body 510 or can be integral therewith. Outlet stem 506 can comprise a tube, such as collection tube 212 (FIG. 2) extending to stone fragment canister 222. In examples, a tube, such as collection tube 212, can be inserted into or disposed around outlet stem 506.

As discussed, it can be desirable to allow for flexibility between a stone fragment captured device and a lithotripsy device to allow for viewing of indicia in a desirable location, but it can be desirable to not have a length of tubing extend from the lithotripsy device to prevent stone fragments from being lodged or stuck within the tubing before reaching the stone fragment capture device. Stone fragment capture device 500 addresses both interests by providing a flexible stone fragment capture device, rather than just a flexible coupling. Stone fragment capture device 500 can comprise a plurality of concentric bodies including tube 502 and filter 508. Tube 502 and filter 508 can comprise flexible bodies thereby allowing the proximal end of stone fragment capture device 500 to change position relative to handpiece 242 (FIG. 4) while the distal end of stone fragment capture device 500 is attached to handpiece 242. Tube 502 can comprise an extension of outlet stem 506 that is widened. In examples, tube 502 can comprise a length of PVC tubing. Filter 508 can comprise a length of a flexible sock or net that can extend within tube 502 along axis AA12. In examples, filter 508 can comprise a length of flexible tubing having openings 514. Thus, filter 508 can allow waste liquid to flow therethrough while stone fragments 522 can remain trapped therein.

Indicia 516 can be provided on one or both of elongate body 510 and filter 508 to allow for measuring of the volume or size of stone fragments 522. Elongate body 510 and filter 508 can be fabricated of transparent material, or material that allows for viewing therethrough, such as mesh, netting or filter material, to allow for viewing of stone fragments 522. In examples, elongate body 510 and filter 508 can comprise flexible transparent tubing and indicia can be provided on elongate body 510. In examples, filter 508 can comprise a filter bag through which stone fragments 522 can be visible, and elongate body 510 can comprise a transparent tube having indicia 516 located thereon. Other combinations can also be used. Due to the flexibility of both tube 502 and filter 508, stone fragment capture device 500 can positioned into an orientation to read indicia 516 regardless of the position of handpiece 242. Indicia 516 can be configured as described herein to provide feedback, assessment, and analysis of the depth, volume, size, color and the like of stone fragments 522, such as by including numerical, textual, symbolic markings or the like.

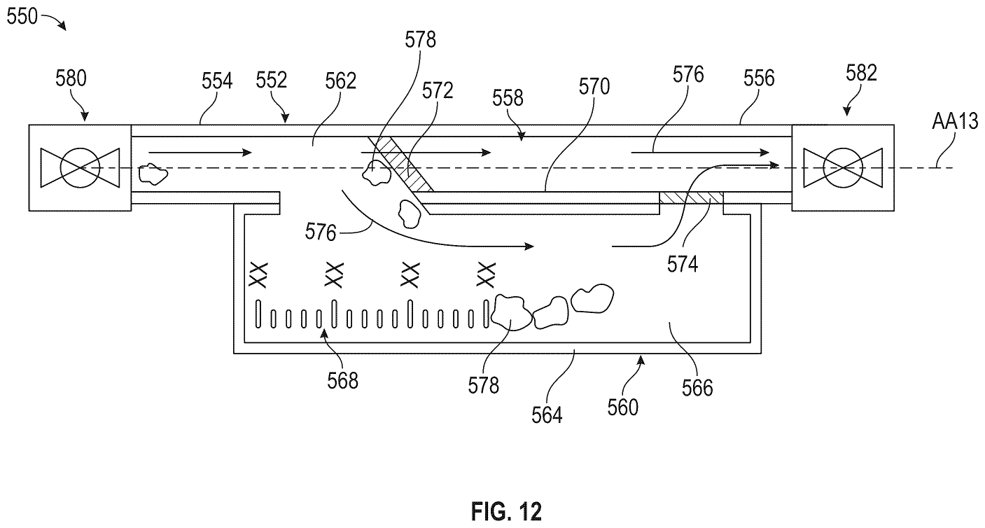

FIG. 12 is a schematic cross-sectional illustration of stone fragment capture device 550 suitable for use with any of the lithotripsy systems described herein, or another surgical system configured to generate suction therethrough. Stone fragment capture device 550 can be used with lithotripsy system 100 of FIG. 1 that can deliver fragmentation energy radially along probe 114, as well as lithotripsy system 200 of FIG. 2 that can deliver fragmentation energy longitudinally along shaft 206.

Stone fragment capture device 550 can comprise tube 552, inlet stem 554 and outlet stem 556. Tube 552 can also comprise filter portion 558 and storage portion 560. Tube 552 can include interior space 562 in which filter portion 558 is positioned. Storage portion 560 can comprise housing 564 extending from tube 552. Storage portion 560 can include internal space 566 in fluid communication with interior space 562. Housing 564 can include indicia 568. Filter portion 558 can comprise interior wall 570, first filter element 572 and second filter element 574. Waste fluid 576, as indicated by arrows, and stone fragments 578 can be positioned within tube 502. Tube 552 can be configured to extend along axis AA13.

Stone fragment capture device 550 can comprise a gravity trap wherein stone fragments 578 can become trapped within storage portion 560 while waste fluid 576 can pass through tube 552. Waste fluid 576 can enter stone fragment capture device 550 at inlet stem 554 after passing through valve 580. Waste fluid 576 can flow into filter portion 558 within tube 502 by passing through first filter element 572. Waste fluid 576 can also flow into internal space 566 and through second filter element 574 to enter filter portion 558. From filter portion 558, waste fluid 576 can leave stone fragment capture device 550 through outlet stem 556 and valve 582. Thus, waste fluid 576 can be allowed to pass through stone fragment capture device 550.

Stone fragments 578 can enter stone fragment capture device 550 at inlet stem 554 after passing through valve 580. In examples, inlet stem 554 can directly receive stem 246 (FIG. 4). Stone fragments 578 can be prevented from flowing into filter portion 558 within tube 502 by first filter element 572. Stone fragments 578 can also flow into internal space 566 and be prevented from flowing into filter portion 558 by second filter element 574. Thus, stone fragments 578 are prevented from leaving stone fragment capture device 550 and passing through outlet stem 556 and valve 582 by first filter element 572 and second filter element 574. As mentioned, gravity can facilitate entry of stone fragments 578 into internal space 566 where stone fragments 578 are out of the way of first filter element 572 to allow waste fluid 576 to pass through first filter element 572 mostly unobstructed.

Storage portion 560 can comprise a housing or container located to a side of tube 552, offset from axis AA13. Stone fragment capture device 550 can be connected to handpiece 242 (FIG. 4) so that storage portion 560 is located downward as a user operates the device. Thus, stone fragments 578 can be pushed downward by gravity into internal space 566. First filter element 572 can be angled to deflect stone fragments 578 toward internal space 566.

Similar to stone fragment capture device 500 of FIG. 11, stone fragment capture device 550 can be configured to flex to facilitate orientating indicia 568 in multiple directions. One or both of tube 552 and storage portion 560 can be configured to flex. The proximal end of stone fragment capture device 550 can thus change position relative to handpiece 242 (FIG. 4) while the distal end of stone fragment capture device 550 can be attached to handpiece 242. Storage portion 560 can be made of transparent material or can include a window of transparent material along which indicia 516 can be located. Indicia 568 can be configured as described herein to provide feedback, assessment, and analysis of the depth, volume, size, color and the like of stone fragments 522, such as by including numerical, textual, graphical, symbolic markings or the like.

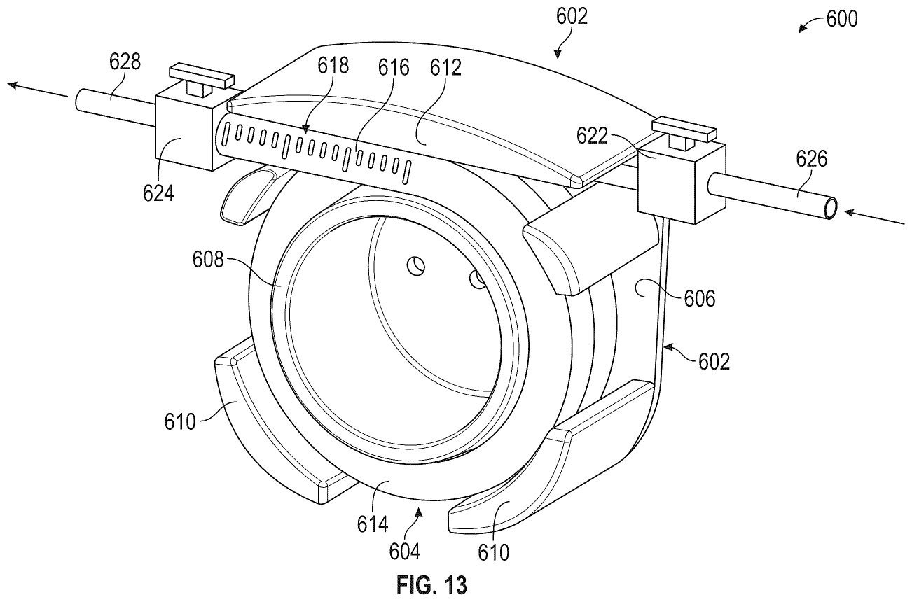

FIG. 13 is a schematic cross-sectional illustration of a stone fragment capture device 600 having bracket 602 and hose 604. Hose 604 can be wound into a coil within bracket 602 to form a helical trap. Bracket 602 can comprise base 606, spool 608, stops 610 and brace 612. Hose 604 can be wound into coil portion 614 and have straight portion 616 including indicia 618. Hose 604 can have ends connected to inlet valve 622 and outlet valve 624. Inlet valve 622 and outlet valve 624 can be connected to tube 626 and tube 628, respectively.

Waste fluid and stone fragments can flow into stone fragment capture device 600 from tube 626, which can comprise a plug that is fluidly connected to handpiece 242 (FIG. 4). The waste fluid and stone fragments can pass through inlet valve 622 and enter hose 604. The waste fluid and stone fragments can enter into coil portion 614 where the stone fragments can gather at the bottom of the coils of hose 604 during use. The weight of the stone fragments can be sufficient to keep the stone fragments at the bottom of coil portion 614 which the waste fluid continues therethrough. The waste fluid can continue into straight portion 616 and continue through outlet valve 624 to enter tube 628. Straight portion 616 can be formed by engagement with brace 612, which can include a flat surface against which hose 604 can be engaged. Tube 628 can comprise a plug or tubing connected to container 232 (FIG. 3) such as collection tube 212 (FIG. 4).

During a procedure, stone fragment capture device 600 can be positioned so that outlet valve 624 is positioned downward and indicia 618 extends longitudinally upward therefrom. Thus, stone fragments within hose 604 can collect at one end of hose 604. Hose 604 can be fabricated of transparent material to facilitate viewing of stone fragments therein. Additionally, after a procedure or during a procedure, hose 604 can be removed from bracket 602 and uncoiled to facilitate movement of stone fragments toward the end of hose 604 having indicia 618. Inlet valve 622 and outlet valve 624 can be closed to seal the stone fragments within hose 604. Inlet valve 622 and outlet valve 624 can comprise any suitable valve such as ball valves, flapper valves, biased stops and the like. In examples, inlet valve 622 and outlet valve 624 can be configured to be biased to closed positions and opened up when tube 626 and tube 628 are inserted therein, respectively. In examples, hose 604 can be cut open to access stone fragments therein after a procedure is finished so the stones can be accessed for laboratory analysis.

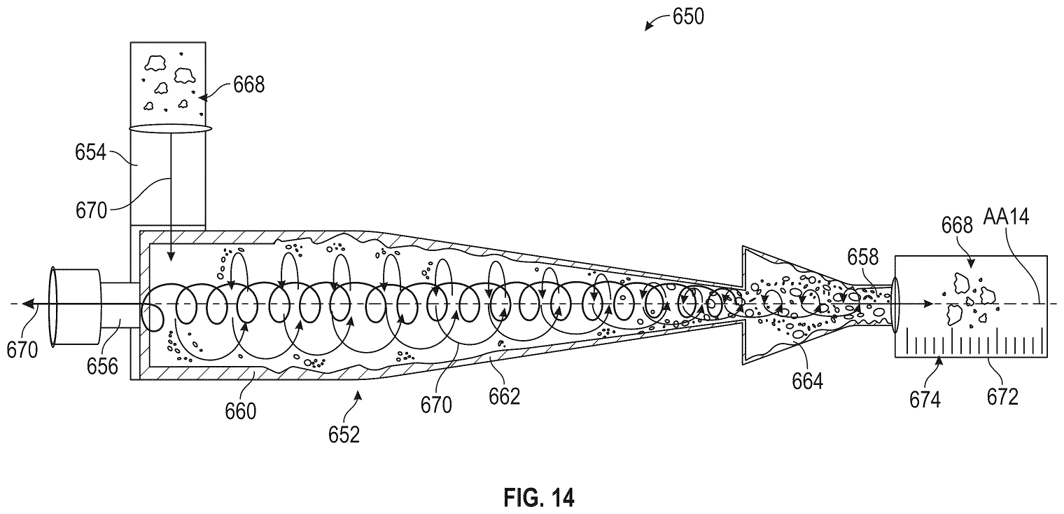

FIG. 14 is a schematic cross-sectional illustration of stone fragment capture device 650 comprising a vortex or cyclonic trap. Stone fragment capture device 650 can comprise housing 652, inlet port 654 and fluid outlet 656 and fragment outlet 658. Housing 652 can comprise cylindrical portion 660, vortex body 662, and fragment trap 664. Housing 652 can extend along axis AA14.

Housing 652 can be disposed so that axis AA14 extends longitudinally. However, stone fragment capture device 650 can operate in other orientations. Tubing or a handle of a lithotripsy device can be connected to inlet port 654 to deliver stone fragments 668 and waste fluid 670 to stone fragment capture device 650. Fluid outlet 656 can be connected to tubing that connects to container 232 (FIG. 3). Fragment outlet 658 be connected to container 672 to collect stone fragments 668. Indicia 674 can be provided on container 672. Stone fragment capture device 650 can be configured to have stone fragments 668 exit stone fragment capture device 650 at fragment outlet 658, while waste fluid separated from stone fragments 668 can exit stone fragment capture device 650 at fluid outlet 656.

In operation, waste fluid 670 and stone fragments 668 can enter cylindrical portion 660. Stone fragments 668 and waste fluid 670 can impact cylindrical portion 660 and can be drawn downward (to the right in FIG. 14) via centrifugal force that can be facilitated by helical channels formed in cylindrical portion 660 and vortex body 662. As waste fluid 670 and stone fragments 668 travel down vortex body, stone fragments 668 can fall into fragment trap 664 due to their larger mass than waste fluid 670 and can be collected in container 672. However, waste fluid 670 can be drawn back upward by the double vortex phenomena, as is known in the art. In examples, auxiliary air, such as clean ambient air, can be introduced into cylindrical portion 660 or vortex body 662 perpendicular to axis AA13 to facilitate generation of vortical flow.

Container 672 can comprise a transparent body or a body having a transparent window through which stone fragments 668 can be viewed. Indicia 674 can be located on container 672 to facilitate measuring or evaluating stone fragments 668 as disclosed herein.

In examples, the indicia, text information, numerical information, symbolic information, etc. described herein and the associated methods of obtaining, determining, assessing and extracting information, such as volume, depth, size, color, etc., from captured stone fragments described herein can be used with the stone fragment capture devices and systems described in Pub. No. US 2022/0047283 A1 to Baker et al. titled “Stone Fragment Capture Systems for Lithotripsy Systems” and which is assigned to Gyrus ACMI, Inc., the contents of which is incorporated herein in its entirety.

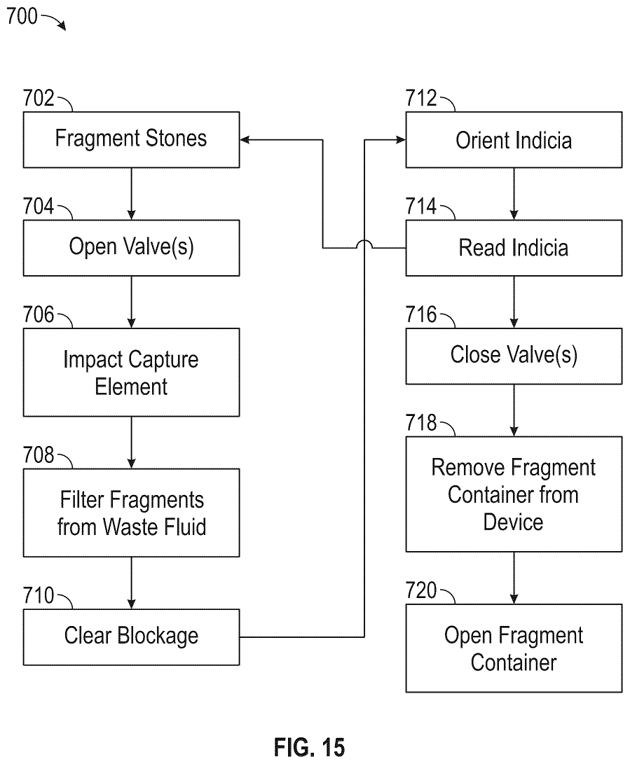

FIG. 15 is a line diagram illustrating method 700 for retrieving stone fragments from a lithotripsy procedure using the devices and stone fragment capture systems of the present disclosure. Method 700 illustrates various exemplary operations and steps of a stone fragment recovery process. Other operations and steps as described herein can be included and some operations and steps can be omitted. Additionally, the illustrated operations steps can be performed in a different order.

At operation 702, stones within a patient can be fragmented using a surgical device, such as one of the lithotripsy devices disclosed herein, e.g., lithotripter 102 and hand-held probe 202. For example, shaft 206 of hand-held probe 202 can be inserted into an incision in a patient and into an anatomical structure, such as a kidney, to reach physiological calculi, e.g., stones, in order to break-up such stones using various forms of energy. Stone fragment capture device 252 of FIG. 6, stone fragment capture device 300 of FIG. 7, stone fragment capture device 350 of FIG. 8A and stone fragment capture device 450 of FIG. 9, stone fragment capture device 400 of FIG. 10, stone fragment capture device 500 of FIG. 11, stone fragment capture device 550 of FIG. 12, stone fragment capture device 600 of FIG. 13, stone fragment capture device 650 of FIG. 14 can be used.

At operation 704, valves, such as an inlet valve (e.g., valve 254 of FIG. 6, valve 332 of FIG. 7, outlet valve 624), of a stone fragment capture device can be opened, such as by the drawing of a vacuum through the medical device used in operation 702. The valve can be opened by insertion of stem 270 (FIG. 6) into the stone fragment capture device. The valve can thus be opened to allow stone fragments and waste fluid into the stone fragment capture device, but can be closed upon removal of stem 270 to prevent exit of the stone fragments from the stone fragment capture device. Stone fragment capture device 252 of FIG. 6, stone fragment capture device 300 of FIG. 7, stone fragment capture device 350 of FIG. 8A and stone fragment capture device 450 of FIG. 9, stone fragment capture device 400 of FIG. 10, stone fragment capture device 500 of FIG. 11, stone fragment capture device 550 of FIG. 12, stone fragment capture device 600 of FIG. 13, stone fragment capture device 650 of FIG. 14 can each be equipped with an inlet valve to allow inflow and prevent outflow, such as into a stone fragment capture area having a filtered outlet.

At operation 706, a stone fragment being pulled by the vacuum being drawn through the stone fragment capture device can engage a capture element, such as a tapered tube (e.g., inlet port 264, inlet socket 304, neck 372), a filter (e.g., filter 256, filter 308, filter 358, filter 458, filter 408, filter 508, first filter element 572, etc.), a valve (e.g., valve 254, valve 332), an orifice (e.g., opening 514) or a baffle (e.g., baffle 267), within the stone fragment capture device. Thus, stone fragments (e.g., stone fragments 284 of FIG. 6, stone fragments 330 of FIG. 7, stone fragments 384 of FIG. 8A, stone fragments 476 of FIG. 9, stone fragments 428 of FIG. 10, stone fragments 522 of FIG. 11, stone fragments 578 of FIG. 12, etc.) can be separated from the flow of waste fluid passing through the stone fragment capture device.

At operation 708, the stone fragment can be diverted out of a main flow of fluid through the stone fragment capture device to be deposited within a container (e.g., container 258, container 302, container 352, container 402, container 452, container 672) of the stone fragment capture device. The stone fragments disposed within the stone fragment capture device can impact a filter (e.g., filter 256, filter 308, filter 358, filter 458, filter 408, etc.) within the stone fragment capture device, thereby only allowing liquid and sufficiently small solids to pass therethrough. Thus, stone fragments (e.g., stone fragments 284 of FIG. 6, stone fragments 330 of FIG. 7, stone fragments 384 of FIG. 8A, stone fragments 476 of FIG. 9, stone fragments 428 of FIG. 10, stone fragments 522 of FIG. 11, stone fragments 578 of FIG. 12, etc.) can be trapped within a housing, container or filter of each of the respective stone fragment capture devices for analysis at operation 714.

At operation 710, a blockage, if any, can be cleared from the surgical device, such as by extending a probe into the stone fragment capture device through a vacuum port, through the stone fragment capture device and into a shaft of the surgical device to clear the blockage.

At operation 712, the stone fragment capture device can be oriented to position captured stone fragments with indicia. In examples, a user can orient the stone fragment capture device to align an axis of the indicia in a vertical orientation. For example, axis AA4 of FIG. 6, axis AA5 or axis AA6 of FIG. 7, axis AA7 of FIG. 8A and axis AA12 of FIG. 11 can be oriented vertically to allow captured stone fragments to fall due to gravity to the bottom of the stone fragment capture device. In examples, axis AA9 of FIG. 9 and axis AA10 of FIG. 10 can be oriented horizontally to allow captured stone fragments to fall due to gravity to the bottom of the stone fragment capture device. The stone fragment capture devices can be shaken or jostled to facilitate settling of the stone fragments to the bottom of the stone fragment capture device and remove empty space between adjacent stone fragments. Thus, stone fragments (e.g., stone fragments 284 of FIG. 6, stone fragments 330 of FIG. 7, stone fragments 384 of FIG. 8A, stone fragments 476 of FIG. 9, stone fragments 428 of FIG. 10, stone fragments 522 of FIG. 11, stone fragments 578 of FIG. 12, etc.) can be disposed in close proximity to indicia (e.g., indicia 290 of FIG. 6, first indicia 316A or second indicia 316B of FIG. 7, indicia 366A or indicia 366B of FIG. 8A, indicia 466 of FIG. 9, indicia 416 of FIG. 10, indicia 516 of FIG. 11, indicia 568 of FIG. 12, indicia 618 of FIG. 13, indicia 674 of FIG. 14) in order to compare the captured stone fragments to information associated with the indicia to extract, obtain or assess information.

At operation 714, the stone fragments can be compared to the indicia to obtain information related to the stone fragments. The indicia can be read to obtain various types of information related to the stone fragments and the procedure being performed. For example, the indicia can provide an indication of the depth (e.g., the total depth of all the captured stone fragments within the stone fragment capture device), volume (e.g., the volume of all the captured stone fragments within the stone fragment capture device), size (e.g., the diameter of a single captured stone fragment within the stone fragment capture device), color (e.g., the shade of color of any of the stone fragments within the stone fragment capture device), and the like. In additional examples, it is contemplated that the indicia can provide feedback regarding texture, density, surface roughness and the like using scales having varying pictures or patterns for comparison to the texture, density and surface roughness of the captured stone fragments.

In examples, the stone fragments (e.g., stone fragments 284 of FIG. 6, stone fragments 330 of FIG. 7, stone fragments 384 of FIG. 8A, stone fragments 476 of FIG. 9, stone fragments 428 of FIG. 10, stone fragments 522 of FIG. 11, stone fragments 578 of FIG. 12, etc.) can be assessed or analyzed to determine if the procedure is progressing as expected. For example, information from the indicia can be used to determine if an adequate volume of stone fragments has been captured. The captured volume can be compared to pre-operative volume to facilitate a determination as to whether or not the procedure being performed has collected all of the stones that were identified pre-operatively. For example, pre-operative imaging can be used to identify stones within a patient, e.g., within a kidney. The volume of stone matter within the imaging can be estimated or determined using manual calculations, 3D modeling, artificial intelligence and the like. Thus, a surgeon or user of the stone fragment capture devices described herein can gain an understanding of whether or not the procedure can continue. If the volume identified in the stone fragment capture device is near, equal or over the pre-operative volume, the procedure can proceed to finish or conclusion at operation 716 onward. If the volume identified in the stone fragment capture device is equal, below or far below the pre-operative volume, the procedure can return to operation 702 or another operation to continue to collect stone fragments until the stone fragment capture device is filled to the desired level. A surgeon can utilize skill and experience to assess the collected volume.

At operation 716, valves, such as an outlet valve (e.g., valve 582, inlet valve 622), of the stone fragment capture device can be closed to retain stone fragments deposited therein. Additionally, valve similar to valve 254 of FIG. 6 or valve 332 of FIG. 7 can be used at any of the outlet passages, outlet ports, outlet stems and the like of stone fragment capture device 252 of FIG. 6, stone fragment capture device 300 of FIG. 7, stone fragment capture device 350 of FIG. 8A and stone fragment capture device 450 of FIG. 9, stone fragment capture device 400 of FIG. 10, stone fragment capture device 500 of FIG. 11, stone fragment capture device 550 of FIG. 12, stone fragment capture device 600 of FIG. 13, stone fragment capture device 650 of FIG. 14 to prevent captured stone fragments from exiting the stone fragment capture device.

At operation 718, the stone fragment capture device holding the stone fragments can be removed from the surgical device. For example, stem 270 (FIG. 6) of hand-held probe 202 can be removed from the inlet port of the stone fragment capture device. Likewise, collection tube 212 (FIG. 4) or tube 276 (FIG. 6) can be removed from the outlet port of the stone fragment capture device.

At operation 720, the container holding the stone fragments can be opened (e.g., by removing a lid or cap, e.g., cap 262, cap 314, lid 364, lid 414, lid 464) such that the stone fragments can be accessed, such as for laboratory analysis.

### VARIOUS NOTES AND EXAMPLES

For the purposes of this disclosure, “proximal” refers to an end of the system that is closer the device operator during use, and “distal” refers to an end of the system that is distal, or further from the device operator during use.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

Example 1 is a stone fragment capture device for a lithotripsy system, the stone fragment capture device comprising: a container for retaining stone fragments; an inlet port in the container for coupling to a suction passage of a lithotripsy device; an outlet port for fluidly coupling to a collection canister of the lithotripsy system; a transparent portion of the container through which stone fragments can be viewed; and indicia on the container for determining a property of stone fragments disposed within the container.

In Example 2, the subject matter of Example 1 optionally includes wherein the indicia comprises graphical, textual, numerical or symbolic indicia of the property of the stone fragments.

In Example 3, the subject matter of any one or more of Examples 1-2 optionally include wherein the property of the stone fragments comprises one or more of depth, volume, size and color.

In Example 4, the subject matter of any one or more of Examples 1-3 optionally include wherein the indicia comprises a graduated scale for determining depth or volume of the stone fragments or a graphical scale for determining color or texture of the stone fragments.

In Example 5, the subject matter of any one or more of Examples 1˜4 optionally include wherein the indicia comprises: a first set of indicia disposed to be upright in a first orientation of the container; and a second set of indicia disposed to be upright in a second orientation of the container different from the first orientation.

In Example 6, the subject matter of any one or more of Examples 1-5 optionally include wherein the inlet port comprises a resilient valve having opposing faces that abut to seal-off the inlet port when not connected to a lithotripsy system.

In Example 7, the subject matter of any one or more of Examples 1-6 optionally include wherein the container comprises a filter disposed between the inlet port and the outlet port.

In Example 8, the subject matter of Example 7 optionally includes wherein: the inlet port comprises a socket for connecting to a hose barb on a handpiece of the lithotripsy device; the socket is axially aligned with the outlet port; and the indicia comprises a graduated scale extending axially between the socket and the outlet port.

In Example 9, the subject matter of any one or more of Examples 7-8 optionally include wherein: the inlet port comprises a socket for connecting to a hose barb on a handpiece of the lithotripsy device; the outlet port is angled relative to the socket; and the indicia comprises: a first graduated scale parallel to the socket; and a second graduated scale parallel to the outlet port.

In Example 10, the subject matter of any one or more of Examples 7-9 optionally include a cap fastened to the container to provide access to an interior of the container, wherein: the filter is attached to the cap; the inlet port and the outlet port connect to the cap along an axis; and the indicia comprises a graduated scale extending perpendicular to the axis.

In Example 11, the subject matter of any one or more of Examples 7-10 optionally include wherein the inlet port comprises a flexible tube.

In Example 12, the subject matter of Example 11 optionally includes wherein the flexible tube is biased to a U-shape.

In Example 13, the subject matter of Example 12 optionally includes a cap fastened to the container to provide access to an interior of the container, wherein the flexible tube extends from the cap and the outlet port extends from the container.

In Example 14, the subject matter of Example 13 optionally includes wherein the U-shape of the flexible tube is made up of segments having differing flexibility.

In Example 15, the subject matter of any one or more of Examples 7-14 optionally include wherein: the container comprises a flexible tube extending from the inlet port; the filter comprises an elongate flexible body extending within the flexible tube; and the indicia comprises a graduated scale extending along one of the flexible tube or the elongate flexible body.

In Example 16, the subject matter of any one or more of Examples 7-15 optionally include wherein: the container comprises a flexible tube extending from the inlet port; the filter comprises an elongate flexible body extending alongside the flexible tube; and the indicia comprises a graduated scale extending along one of the flexible tube or the elongate flexible body.

In Example 17, the subject matter of any one or more of Examples 1-16 optionally include wherein the container comprises a coil of tubing configured to trap stone fragments.

In Example 18, the subject matter of Example 17 optionally includes a spool around which the coil of tubing is wound; a first plug for fluidly connecting to tubing, the first plug comprising the inlet port; and a second plug for fluidly connecting to tubing, the second plug comprising the outlet port.

In Example 19, the subject matter of any one or more of Examples 1-18 optionally include wherein the container comprises a cyclonic trap.

Example 20 is a lithotripsy device comprising: a handpiece configured to be held by a user; an energization source configured to generate an energy for breaking apart a physiological calculi; a shaft having a proximal end extending from the handpiece and a distal end configured to engage the physiological calculi; and a stone fragment capture device fluidly connected to the handpiece, the stone fragment capture device comprising: a container into which waste fluid and stone fragments from the handpiece can flow; a trap element connected to the container to extract the stone fragments from flow of the waste fluid; and indicia provided on the stone fragment capture device for comparison to stone fragments within the stone fragment capture device.

In Example 21, the subject matter of Example 20 optionally includes wherein the trap element comprises a filter.

In Example 22, the subject matter of any one or more of Examples 20-21 optionally include wherein the indicia comprises a graduated scale.

In Example 23, the subject matter of any one or more of Examples 20-22 optionally include wherein the indicia can be used to determine a depth, volume, size, texture, smoothness or color of the stone fragments.

In Example 24, the subject matter of any one or more of Examples 20-23 optionally include wherein the container includes at least a portion of transparent material to allow viewing of the stone fragments within the container.

In Example 25, the subject matter of any one or more of Examples 20-24 optionally include wherein the container comprises a rigid housing directly connected to the handpiece such that stone fragments can enter the container directly from the handpiece.

In Example 26, the subject matter of any one or more of Examples 20-25 optionally include a flexible tube connecting the container to the handpiece, wherein the flexible tube comprises: an inlet segment; an outlet segment; and a middle segment connecting the inlet segment and the outlet segment; wherein the middle segment is angled relative to the inlet segment and the outlet segment.

In Example 27, the subject matter of any one or more of Examples 20-26 optionally include wherein the container comprises a flexible housing directly connected to the handpiece such that stone fragments can enter the container directly from the handpiece, wherein the trap element comprises a flexible filter positioned within or alongside the flexible housing.

In Example 28, the subject matter of any one or more of Examples 20-27 optionally include wherein the container comprises a helical or vortical stone fragment trap.

Example 29 is a method of retrieving stone fragments from a lithotripsy procedure, the method comprising: fragmenting stones with a lithotripsy device; drawing a vacuum through the lithotripsy device to pull stone fragments and waste fluid through the lithotripsy device; pulling the vacuum through a stone fragment capture device connected to the lithotripsy device; separating the stone fragments from the waste fluid within the stone fragment capture device; determining a property of the stone fragments within the stone fragment capture device; and finishing the lithotripsy procedure.

In Example 30, the subject matter of Example 29 optionally includes wherein determining the property of the stone fragments comprises measuring a volume of the stone fragments within the stone fragment capture device.

In Example 31, the subject matter of Example 30 optionally includes filling the stone fragment capture device to a desired volume after measuring the volume of the stone fragments.

In Example 32, the subject matter of any one or more of Examples 29-31 optionally include orienting the stone fragment capture device in a first position to align volume indicia with the stone fragments.

In Example 33, the subject matter of Example 32 optionally includes wherein the first position is when a shaft of the lithotripsy device is in a horizontal position.

In Example 34, the subject matter of Example 33 optionally includes orienting the stone fragment capture device in a second position to align volume indicia with the stone fragments.

In Example 35, the subject matter of Example 34 optionally includes wherein the first position is when a shaft of the lithotripsy device is in a horizontal position.

In Example 36, the subject matter of any one or more of Examples 29-35 optionally include opening the stone fragment capture device to access deposited stone fragments after finishing the lithotripsy procedure.

Each of these non-limiting examples can stand on its own, or can be combined in various permutations or combinations with one or more of the other examples.

What is claimed is:

1. A stone fragment capture device for a lithotripsy system, the stone fragment capture device comprising:
a container for retaining stone fragments;
an inlet port in the container for coupling to a suction passage of a lithotripsy device;
an outlet port for fluidly coupling to a collection canister of the lithotripsy system;
a transparent portion of the container through which stone fragments can be viewed; and
indicia on the container for determining a property of stone fragments disposed within the container.

2. The stone fragment capture device of claim 1, wherein the indicia comprises graphical, textual, numerical or symbolic indicia of the property of the stone fragments, wherein the property of the stone fragments comprises one or more of depth, volume, size and color.

3. The stone fragment capture device of claim 1, wherein the indicia comprises a graduated scale for determining depth or volume of the stone fragments or a graphical scale for determining color or texture of the stone fragments.

4. The stone fragment capture device of claim 1, wherein the indicia comprises:
a first set of indicia disposed to be upright in a first orientation of the container; and
a second set of indicia disposed to be upright in a second orientation of the container different from the first orientation.

5. The stone fragment capture device of claim 1, wherein the inlet port comprises a resilient valve having opposing faces that abut to seal-off the inlet port when not connected to a lithotripsy system.

6. The stone fragment capture device of claim 1, wherein the container comprises a filter disposed between the inlet port and the outlet port.

7. The stone fragment capture device of claim 6, wherein:
the inlet port comprises a socket for connecting to a hose barb on a handpiece of the lithotripsy device;
the socket is axially aligned with the outlet port; and
the indicia comprises a graduated scale extending axially between the socket and the outlet port.

8. The stone fragment capture device of claim 6, wherein:
the inlet port comprises a socket for connecting to a hose barb on a handpiece of the lithotripsy device;
the outlet port is angled relative to the socket; and
the indicia comprises:
a first graduated scale parallel to the socket; and
a second graduated scale parallel to the outlet port.

9. The stone fragment capture device of claim 6, further comprising a cap fastened to the container to provide access to an interior of the container, wherein:
the filter is attached to the cap;
the inlet port and the outlet port connect to the cap along an axis; and
the indicia comprises a graduated scale extending perpendicular to the axis.

10. The stone fragment capture device of claim 6, wherein the inlet port comprises a flexible tube.

11. The stone fragment capture device of claim 10, wherein the flexible tube is biased to a U-shape.

12. The stone fragment capture device of claim 11, further comprising a cap fastened to the container to provide access to an interior of the container, wherein the flexible tube extends from the cap and the outlet port extends from the container.

13. The stone fragment capture device of claim 12, wherein the U-shape of the flexible tube is made up of segments having differing flexibility.

14. The stone fragment capture device of claim 6, wherein:
the container comprises a flexible tube extending from the inlet port;
the filter comprises an elongate flexible body extending within the flexible tube; and
the indicia comprises a graduated scale extending along one of the flexible tube or the elongate flexible body.

15. The stone fragment capture device of claim 6, wherein:
the container comprises a flexible tube extending from the inlet port;
the filter comprises an elongate flexible body extending alongside the flexible tube; and
the indicia comprises a graduated scale extending along one of the flexible tube or the elongate flexible body.

16. The stone fragment capture device of claim 1, wherein the container comprises a coil of tubing configured to trap stone fragments.

17. The stone fragment capture device of claim 16, further comprising:
a spool around which the coil of tubing is wound;
a first plug for fluidly connecting to tubing, the first plug comprising the inlet port; and
a second plug for fluidly connecting to tubing, the second plug comprising the outlet port.

18. The stone fragment capture device of claim 1, wherein the container comprises a cyclonic trap.

19. A lithotripsy device comprising:
a handpiece configured to be held by a user;
an energization source configured to generate an energy for breaking apart a physiological calculi;
a shaft having a proximal end extending from the handpiece and a distal end configured to engage the physiological calculi; and
a stone fragment capture device fluidly connected to the handpiece, the stone fragment capture device comprising:
a container into which waste fluid and stone fragments from the handpiece can flow;
a trap element connected to the container to extract the stone fragments from flow of the waste fluid; and
indicia provided on the stone fragment capture device for comparison to stone fragments within the stone fragment capture device.

20. A method of retrieving stone fragments from a lithotripsy procedure, the method comprising:
fragmenting stones with a lithotripsy device;
drawing a vacuum through the lithotripsy device to pull stone fragments and waste fluid through the lithotripsy device;
pulling the vacuum through a stone fragment capture device connected to the lithotripsy device;
separating the stone fragments from the waste fluid within the stone fragment capture device;
determining a property of the stone fragments within the stone fragment capture device; and
finishing the lithotripsy procedure.
